(19) United States
(12) Patent Application Publication
(10) Pub. No.: US 2025/0375441 A1
(43) Pub. Date: Dec. 11, 2025

(54) ONCOLYTIC THERAPY WITH A DUAL HDAC3 AND HDAC8 TARGETING AGENT

(71) Applicant: University of Florida Research Foundation, Incorporated, Gainesville, FL (US)

(72) Inventors: Beth A. Rousseau, Gainesville, FL (US); Sumita Bhaduri-Mclntosh, Gainesville, FL (US); Michael Mclntosh, Gainesville, FL (US); Guangrong Zheng, Gainesville, FL (US); Yufeng Xiao, Gainesville, FL (US)

(73) Assignee: University of Florida Research Foundation, Incorporated, Gainesville, FL (US)

(21) Appl. No.: 19/229,044

(22) Filed: Jun. 5, 2025

Related U.S. Application Data
(60) Provisional application No. 63/656,775, filed on Jun. 6, 2024.

(51) Int. Cl. A61K 31/496; A61K 31/522; A61K 31/662; A61P 31/22

(52) U.S. Cl. CPC A61K 31/496; A61K 31/522; A61K 31/662; A61P 31/22

(57) ABSTRACT

The present disclosure provides methods of treating or preventing a disease associated with a virus, methods of inhibiting cell proliferation or promoting apoptosis, methods of inducing a lytic phase of a virus, and methods of reactivating a virus by administering to a subject in need thereof a dual HDAC3 and HDAC8 targeting agent and an antiviral, or pharmaceutically acceptable salts thereof, or pharmaceutical compositions thereof.

### CROSS-REFERENCE TO RELATED APPLICATIONS

This application claims the benefit of and priority under 35 U.S.C. §119(e) to U.S. Provisional Application Ser. No. 63/656,775, filed Jun. 6, 2024, the contents of which are incorporated herewith by reference in their entirety.

### BACKGROUND OF THE INVENTION

Epstein-Barr virus (EBV) is a human herpesvirus that infects most humans. It is one of two members of the gamma-herpesvirus family known to cause cancer in humans; the other member is Kaposi's Sarcoma-associated herpesvirus (KSHV), which is similar in many respects to EBV. EBV infects B cells of the immune system and epithelial cells. Once EBV's initial lytic infection is brought under control, EBV latency persists in the individual's B cells for the rest of their life. Most people become infected with EBV and gain adaptive immunity. In the United States, about half of all five-year-old children and about 90% of adults have evidence of previous EBV infection.

EBV is associated with various non-malignant, premalignant, and malignant lymphoproliferative diseases such as Burkitt lymphoma, diffuse large B-cell lymphoma (DLBCL), hemophagocytic lymphohistiocytosis, and Hodgkin's lymphoma; non-lymphoid malignancies such as gastric cancer and nasopharyngeal carcinoma; and conditions associated with human immunodeficiency virus such as oral hairy leukoplakia and central nervous system lymphomas (Kosowicz et al., J. Virology 2017. “Drug Modulators of B Cell Signaling Pathways and Epstein-Barr Virus Lytic Activation”).

EBV has two modes of infection: latent and lytic. The lytic cycle, or productive phase, results in the production of infectious virions. EBV infection is predominantly latent, and malignancies can arise from latently infected cells. The switch from latency to the lytic cycle is known as EBV activation or EBV reactivation.

Oncolytic therapies destroy cancer cells by purposefully reactivating the lytic phase of EBV followed by cancer cell death through the genomic incorporation of an antiviral, such as the small molecule ganciclovir. For ganciclovir genomic incorporation to occur, it must first be activated by a lytic phase viral protein. Ganciclovir also blocks the production of new virions of EBV by inhibiting replication of the viral genome. Molecules that induce the lytic phase of EBV include HDAC inhibitors, DNA methyltransferase inhibitors, phorbol esters, drugs that mimic hypoxia, and immunoglobulins. Sodium butyrate, valproic acid, romidepsin, MS-275, apicidin, oxamflatin, Scriptaid, panobinostat, nanatinostat are a few well-characterized HDAC inhibitors that reactivate EBV. However, oncolytic clinical trials for EBV cancers with some of these agents have previously failed due to off target effects, since these agents are generally broad acting with very little selectivity. Thus, there is a need for agents that reactivate EBV with selective modes of function.

### SUMMARY OF THE INVENTION

The present disclosure discloses that selective targeting (e.g., degradation) of HDAC3 and HDAC8 by a small molecule robustly reactivates EBV in cancer cells. It is known that cancer cells harboring latent EBV will die after lytic EBV reactivation and exposure to a small molecule that blocks DNA replication by incorporating into cell and viral genomes (e.g., ganciclovir)-the so-called oncolytic therapy. This process also ensures that any newly formed EBV virions are incapable of infecting new cells, as the genomes are likely to be defective due to incomplete replication. As such, the combined therapy of a small molecule which selectively targets (e.g., degrades) HDAC3 and HDAC8 with an antiviral such as ganciclovir may be an effective and specific therapy which targets cells infected with EBV but not healthy, uninfected cells, allowing cancer patients to be put into remission.

Accordingly, in one aspect, the present disclosure provides a method of treating or preventing a disease associated with a virus in a subject in need thereof, the method comprising administering to the subject in need thereof a therapeutically effective amount of a dual HDAC3 and HDAC8 targeting agent, or a pharmaceutically acceptable salt thereof.

In another aspect, the present disclosure provides a method of treating or preventing a disease associated with a virus in a subject in need thereof, the method comprising administering to the subject in need thereof:

a therapeutically effective amount of a dual HDAC3 and HDAC8 targeting agent, or a pharmaceutically acceptable salt thereof; and

a therapeutically effective amount of an antiviral agent, or a pharmaceutically acceptable salt thereof.

In another aspect, the present disclosure provides a method of treating or preventing a disease associated with a virus in a subject in need thereof, the method comprising administering to the subject in need thereof:

a therapeutically effective amount of a dual HDAC3 and HDAC8 targeting agent, or a pharmaceutically acceptable salt thereof; and

a therapeutically effective amount of an agent capable of sensitizing or potentiating induction of the lytic phase of the virus, or a pharmaceutically acceptable salt thereof.

In another aspect, the present disclosure provides a method of treating or preventing a disease associated with a virus in a subject in need thereof, the method comprising administering to the subject in need thereof a pharmaceutical composition comprising:

a therapeutically effective amount of a dual HDAC3 and HDAC8 targeting agent, or a pharmaceutically acceptable salt thereof; and

a pharmaceutically acceptable excipient.

In another aspect, the present disclosure provides a method of treating or preventing a disease associated with a virus in a subject in need thereof, the method comprising administering to the subject in need thereof a pharmaceutical composition comprising:

a therapeutically effective amount of a dual HDAC3 and HDAC8 targeting agent, or a pharmaceutically acceptable salt thereof;

a therapeutically effective amount of an antiviral agent, or a pharmaceutically acceptable salt thereof; and

a pharmaceutically acceptable excipient.

In another aspect, the present disclosure provides a method of treating or preventing a disease associated with a virus in a subject in need thereof, the method comprising administering to the subject in need thereof a pharmaceutical composition comprising:

a therapeutically effective amount of a dual HDAC3 and HDAC8 targeting agent, or a pharmaceutically acceptable salt thereof;

a therapeutically effective amount of an agent capable of sensitizing or potentiating induction of the lytic phase of the virus, or a pharmaceutically acceptable salt thereof; and

a pharmaceutically acceptable excipient.

In another aspect, the present disclosure provides a method of inhibiting cell proliferation or promoting apoptosis in a subject in need thereof, the method comprising administering to the subject in need thereof an effective amount of a dual HDAC3 and HDAC8 targeting agent, or a pharmaceutically acceptable salt thereof.

In another aspect, the present disclosure provides a method of inducing a lytic phase of a virus in a subject in need thereof, the method comprising administering to the subject in need thereof an effective amount of a dual HDAC3 and HDAC8 targeting agent, or a pharmaceutically acceptable salt thereof.

In another aspect, the present disclosure provides a method of reactivating a virus in a subject in need thereof, the method comprising administering to the subject in need thereof an effective amount of a dual HDAC3 and HDAC8 targeting agent, or a pharmaceutically acceptable salt thereof.

In another aspect, the present disclosure provides a method of inhibiting cell proliferation or promoting apoptosis in a subject in need thereof, the method comprising administering to the subject in need thereof a pharmaceutical composition comprising:

an effective amount of a dual HDAC3 and HDAC8 targeting agent, or a pharmaceutically acceptable salt thereof; and

a pharmaceutically acceptable excipient.

In another aspect, the present disclosure provides a method of inducing a lytic phase of a virus in a subject in need thereof, the method comprising administering to the subject in need thereof a pharmaceutical composition comprising:

an effective amount of a dual HDAC3 and HDAC8 targeting agent, or a pharmaceutically acceptable salt thereof; and

a pharmaceutically acceptable excipient.

In another aspect, the present disclosure provides a method of reactivating a virus in a subject in need thereof, the method comprising administering to the subject in need thereof a pharmaceutical composition comprising:

an effective amount of a dual HDAC3 and HDAC8 targeting agent, or a pharmaceutically acceptable salt thereof; and

a pharmaceutically acceptable excipient.

In another aspect, the present disclosure provides a kit comprising:

a therapeutically effective amount of a dual HDAC3 and HDAC8 targeting agent, or a pharmaceutically acceptable salt thereof; and

instructions for its use.

In another aspect, the present disclosure provides a kit comprising:

a pharmaceutical composition comprising:
a therapeutically effective amount of a dual HDAC3 and HDAC8 targeting agent, or a pharmaceutically acceptable salt thereof; and
a therapeutically effective amount of an antiviral agent, or a pharmaceutically acceptable salt thereof; and

instructions for its use.

In another aspect, the present disclosure provides a kit comprising:

a pharmaceutical composition comprising:
a therapeutically effective amount of a dual HDAC3 and HDAC8 targeting agent, or a pharmaceutically acceptable salt thereof; and
a therapeutically effective amount of an agent capable of sensitizing or potentiating induction of the lytic phase of the virus, or a pharmaceutically acceptable salt thereof; and

instructions for its use.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

FIG. 1 shows flow plots showing that YX968 induces the lytic phase in Burkitt lymphoma cells (HH514-16).

FIG. 2 shows a qPCR graph showing that the late EBV gene BFRF3 is upregulated in YX968 induced in Burkitt lymphoma cells.

FIG. 3 shows that Burkitt lymphoma cells die after exposure to YX968.

FIG. 4 shows that ganciclovir inhibits the replication of the EBV genome after the lytic cycle has been started by YX968.

FIG. 5 shows that KSHV RTA transcripts increase after treatment with YX968.

FIGS. 6A-6B show that overexpression of HDAC2 (FIG. 6B) causes a decrease in the percentage of lytic cells induced by YX968, whereas overexpression of HDAC1 does not (FIG. 6A).

FIG. 7 shows that acetylation of H3K27 increases at the BZLF1 promoter post-addition of YX968.

FIG. 8 shows that combining YX968 and tazemetostat leads to a greater increase in lytic cells than treatment with YX968 alone.

### DEFINITIONS

When a range of values (“range”) is listed, it encompasses each value and sub-range within the range. A range is inclusive of the values at the two ends of the range unless otherwise provided.

The term “about X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the present disclosure is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the present disclosure are administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inducing the lytic phase of a virus (e.g., Epstein-Barr virus (EBV), Kaposi's sarcoma-associated herpesvirus (KSHV)). In certain embodiments, a therapeutically effective amount is an amount sufficient for treating a disease associated with a virus (e.g., a disease associated with Epstein-Barr virus (EBV), Kaposi's sarcoma-associated herpesvirus (KSHV)). In certain embodiments, a therapeutically effective amount is an amount sufficient for inducing the lytic phase of a virus (e.g., Epstein-Barr virus (EBV), Kaposi's sarcoma-associated herpesvirus (KSHV)) and treating a disease associated with a virus (e.g., a disease associated with Epstein-Barr virus (EBV), Kaposi's sarcoma-associated herpesvirus (KSHV)).

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inducing the lytic phase of a virus (e.g., Epstein-Barr virus (EBV), Kaposi's sarcoma-associated herpesvirus (KSHV)). In certain embodiments, a prophylactically effective amount is an amount sufficient for preventing a disease associated with a virus (e.g., a disease associated with Epstein-Barr virus (EBV), Kaposi's sarcoma-associated herpesvirus (KSHV)). In certain embodiments, a prophylactically effective amount is an amount sufficient for inducing the lytic phase of a virus (e.g., Epstein-Barr virus (EBV), Kaposi's sarcoma-associated herpesvirus (KSHV)) and preventing a disease associated with a virus (e.g., a disease associated with Epstein-Barr virus (EBV), Kaposi's sarcoma-associated herpesvirus (KSHV)).

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The term “inhibit” or “inhibition” in the context of enzymes refers to a reduction in the activity of the enzyme. In some embodiments, the term refers to a reduction of the level of enzyme activity to a level that is statistically significantly lower than an initial level, which may, for example, be a baseline level of enzyme activity. In some embodiments, the term refers to a reduction of the level of enzyme activity to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g.,bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Anti-cancer agents encompass biotherapeutic anti-cancer agents as well as chemotherapeutic agents.

Exemplary biotherapeutic anti-cancer agents include, but are not limited to, interferons, cytokines (e.g., tumor necrosis factor, interferon a, interferon Y), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g., HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), platinum containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., ‘2’-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca2+ ATPase inhibitors (e.g., thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOKTM), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin,, aminopterin, and hexamethyl melamine.

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, hemolytic autoimmune anemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenia purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischemic colitis, diversion colitis, Behcet's syndrome, indeterminate colitis) and inflammatory bowel syndrome (IBS)), lupus, multiple sclerosis, morphea, myasthenia gravis, myocardial ischemia, nephrotic syndrome, pemphigus vulgaris, pernicious anemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, scleroderma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatitis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopeni purpura and disseminated intravascular coagulation, infections by parasites such as Plasmodium, chemical injuries from, e.g., lead poisoning, and hypersplenism.

Immune disorders, such as auto-immune disorders, include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

“Immunosuppressive therapy” and “immunomodulatory therapy” are drug regimens that are used to lower or modulate the activity of a patient's immune system. The drug regiments prevent the immune system from overreacting and rejecting or damaging transplanted organs and tissues. Most transplant patients receive immunosuppressive therapy or immunomodulatory therapy when receiving a transplant. Immunosuppressive therapy and immunomodulatory therapy may also be used to treat conditions in which the immune system is overactive, such as autoimmune diseases and allergies. Some types of immunosuppressive therapy may increase a person's risk of cancer by lowering the body's ability to kill cancer cells. Persons with lymphoproliferative diseases may benefit from oncolytic therapy.

“YX968” refers to a dual HDAC3/HDAC8 degrader (CAS: 2742732-92-7) of formula:

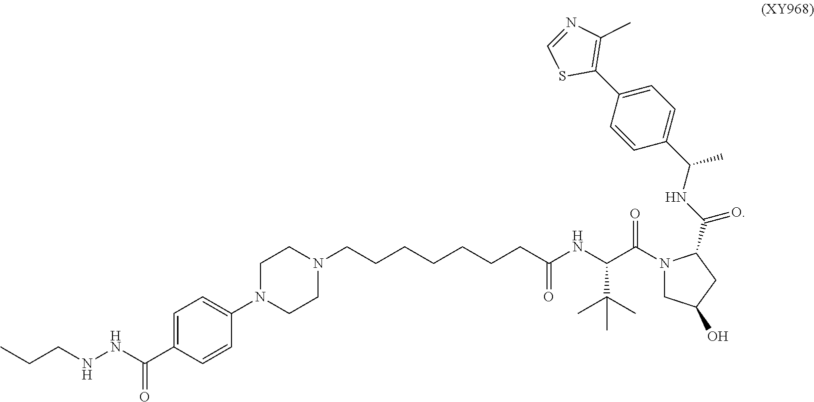 [SMILES: CCCNNC(=O)c1ccc(N2CCN(CCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Methods of Treatment and Prevention

In one aspect, the present disclosure provides a method of treating or preventing a disease associated with a virus in a subject in need thereof, the method comprising administering to the subject in need thereof a therapeutically effective amount of a dual HDAC3 and HDAC8 targeting agent, or a pharmaceutically acceptable salt thereof. In some embodiments, the present disclosure provides a method of treating a disease associated with a virus in a subject in need thereof, the method comprising administering to the subject in need thereof a therapeutically effective amount of a dual HDAC3 and HDAC8 targeting agent, or a pharmaceutically acceptable salt thereof. In some embodiments, the present disclosure provides a method of preventing a disease associated with a virus in a subject in need thereof, the method comprising administering to the subject in need thereof a therapeutically effective amount of a dual HDAC3 and HDAC8 targeting agent, or a pharmaceutically acceptable salt thereof.

In some embodiments, the method further comprises administering to the subject in need thereof a therapeutically effective amount of an antiviral agent, or a pharmaceutically acceptable salt thereof.

In some embodiments, the method further comprises administering to the subject in need thereof a therapeutically effective amount of an agent capable of sensitizing or potentiating induction of the lytic phase of the virus, or a pharmaceutically acceptable salt thereof.

In some embodiments, the dual HDAC3 and HDAC8 targeting agent is administered to the subject in need thereof after the subject in need thereof has been diagnosed as comprising malignant or pre-malignant cells infected with the virus. In some embodiments, the dual HDAC3 and HDAC8 targeting agent is administered to the subject in need thereof after the subject in need thereof has been diagnosed as comprising malignant cells infected with the virus. In some embodiments, the dual HDAC3 and HDAC8 targeting agent is administered to the subject in need thereof after the subject in need thereof has been diagnosed as comprising pre-malignant cells infected with the virus. In some embodiments, the virus is in the latent state or the abortive lytic state. In some embodiments, the virus is in the latent state. In some embodiments, the virus is in the abortive lytic state.

In another aspect, the present disclosure provides a method of treating or preventing a disease associated with a virus in a subject in need thereof, the method comprising administering to the subject in need thereof: a therapeutically effective amount of a dual HDAC3 and HDAC8 targeting agent, or a pharmaceutically acceptable salt thereof; and a therapeutically effective amount of an antiviral agent, or a pharmaceutically acceptable salt thereof. In another aspect, the present disclosure provides a method of treating a disease associated with a virus in a subject in need thereof, the method comprising administering to the subject in need thereof: a therapeutically effective amount of a dual HDAC3 and HDAC8 targeting agent, or a pharmaceutically acceptable salt thereof; and a therapeutically effective amount of an antiviral agent, or a pharmaceutically acceptable salt thereof. In another aspect, the present disclosure provides a method of preventing a disease associated with a virus in a subject in need thereof, the method comprising administering to the subject in need thereof: a therapeutically effective amount of a dual HDAC3 and HDAC8 targeting agent, or a pharmaceutically acceptable salt thereof; and a therapeutically effective amount of an antiviral agent, or a pharmaceutically acceptable salt thereof.

In another aspect, the present disclosure provides a method of treating or preventing a disease associated with a virus in a subject in need thereof, the method comprising administering to the subject in need thereof: a therapeutically effective amount of a dual HDAC3 and HDAC8 targeting agent, or a pharmaceutically acceptable salt thereof; and a therapeutically effective amount of an agent capable of sensitizing or potentiating induction of the lytic phase of the virus, or a pharmaceutically acceptable salt thereof. In another aspect, the present disclosure provides a method of treating a disease associated with a virus in a subject in need thereof, the method comprising administering to the subject in need thereof: a therapeutically effective amount of a dual HDAC3 and HDAC8 targeting agent, or a pharmaceutically acceptable salt thereof; and a therapeutically effective amount of an agent capable of sensitizing or potentiating induction of the lytic phase of the virus, or a pharmaceutically acceptable salt thereof. In another aspect, the present disclosure provides a method of preventing a disease associated with a virus in a subject in need thereof, the method comprising administering to the subject in need thereof: a therapeutically effective amount of a dual HDAC3 and HDAC8 targeting agent, or a pharmaceutically acceptable salt thereof; and a therapeutically effective amount of an agent capable of sensitizing or potentiating induction of the lytic phase of the virus, or a pharmaceutically acceptable salt thereof.

In some embodiments, the dual HDAC3 and HDAC8 targeting agent is administered to the subject in need thereof before the antiviral agent is administered to the subject in need thereof. In some embodiments, the dual HDAC3 and HDAC8 targeting agent is administered to the subject in need thereof concurrently with the antiviral agent.

In some embodiments, the dual HDAC3 and HDAC8 targeting agent is administered to the subject in need thereof after the agent capable of sensitizing or potentiating induction of the lytic phase of the virus, or pharmaceutically acceptable salt thereof, is administered to the subject in need thereof. In some embodiments, the dual HDAC3 and HDAC8 targeting agent is administered to the subject in need thereof concurrently with the agent capable of sensitizing or potentiating induction of the lytic phase of the virus, or pharmaceutically acceptable salt thereof.

In some embodiments, the dual HDAC3 and HDAC8 targeting agent is administered to the subject in need thereof after the subject in need thereof has been diagnosed as comprising malignant or pre-malignant cells infected with the virus. In some embodiments, the dual HDAC3 and HDAC8targeting agent is administered to the subject in need thereof after the subject in need thereof has been diagnosed as comprising malignant cells infected with the virus. In some embodiments, the dual HDAC3and HDAC8 targeting agent is administered to the subject in need thereof after the subject in need thereof has been diagnosed as comprising pre-malignant cells infected with the virus. In some embodiments, the virus is in the latent state or the abortive lytic state. In some embodiments, the virus is in the latent state. In some embodiments, the virus is in the abortive lytic state.

In another aspect, the present disclosure provides a method of treating or preventing a disease associated with a virus in a subject in need thereof, the method comprising administering to the subject in need thereof a pharmaceutical composition comprising: a therapeutically effective amount of a dual HDAC3 and HDAC8 targeting agent, or a pharmaceutically acceptable salt thereof; and a pharmaceutically acceptable excipient. In another aspect, the present disclosure provides a method of treating a disease associated with a virus in a subject in need thereof, the method comprising administering to the subject in need thereof a pharmaceutical composition comprising: a therapeutically effective amount of a dual HDAC3 and HDAC8 targeting agent, or a pharmaceutically acceptable salt thereof; and a pharmaceutically acceptable excipient. In another aspect, the present disclosure provides a method of preventing a disease associated with a virus in a subject in need thereof, the method comprising administering to the subject in need thereof a pharmaceutical composition comprising: a therapeutically effective amount of a dual HDAC3 and HDAC8 targeting agent, or a pharmaceutically acceptable salt thereof; and a pharmaceutically acceptable excipient.

In some embodiments, the pharmaceutical composition further comprises a therapeutically effective amount of an antiviral agent, or a pharmaceutically acceptable salt thereof.

In some embodiments, the pharmaceutical composition further comprises a therapeutically effective amount of an agent capable of sensitizing or potentiating induction of the lytic phase of the virus, or a pharmaceutically acceptable salt thereof.

In some embodiments, the pharmaceutical composition is administered to the subject in need thereof after the subject in need thereof has been diagnosed as comprising malignant or pre-malignant cells infected with the virus. In some embodiments, the pharmaceutical composition is administered to the subject in need thereof after the subject in need thereof has been diagnosed as comprising malignant cells infected with the virus. In some embodiments, the pharmaceutical composition is administered to the subject in need thereof after the subject in need thereof has been diagnosed as comprising pre-malignant cells infected with the virus. In some embodiments, the virus is in the latent state or the abortive lytic state. In some embodiments, the virus is in the latent state. In some embodiments, the virus is in the abortive lytic state.

In another aspect, the present disclosure provides a method of treating or preventing a disease associated with a virus in a subject in need thereof, the method comprising administering to the subject in need thereof a pharmaceutical composition comprising: a therapeutically effective amount of a dual HDAC3 and HDAC8 targeting agent, or a pharmaceutically acceptable salt thereof; a therapeutically effective amount of an antiviral agent, or a pharmaceutically acceptable salt thereof; and a pharmaceutically acceptable excipient. In another aspect, the present disclosure provides a method of treating a disease associated with a virus in a subject in need thereof, the method comprising administering to the subject in need thereof a pharmaceutical composition comprising: a therapeutically effective amount of a dual HDAC3 and HDAC8 targeting agent, or a pharmaceutically acceptable salt thereof; a therapeutically effective amount of an antiviral agent, or a pharmaceutically acceptable salt thereof; and a pharmaceutically acceptable excipient. In another aspect, the present disclosure provides a method of preventing a disease associated with a virus in a subject in need thereof, the method comprising administering to the subject in need thereof a pharmaceutical composition comprising: a therapeutically effective amount of a dual HDAC3 and HDAC8 targeting agent, or a pharmaceutically acceptable salt thereof; a therapeutically effective amount of an antiviral agent, or a pharmaceutically acceptable salt thereof; and a pharmaceutically acceptable excipient.

In another aspect, the present disclosure provides a method of treating or preventing a disease associated with a virus in a subject in need thereof, the method comprising administering to the subject in need thereof a pharmaceutical composition comprising: a therapeutically effective amount of a dual HDAC3 and HDAC8 targeting agent, or a pharmaceutically acceptable salt thereof; a therapeutically effective amount of an agent capable of sensitizing or potentiating induction of the lytic phase of the virus, or a pharmaceutically acceptable salt thereof; and a pharmaceutically acceptable excipient. In another aspect, the present disclosure provides a method of treating a disease associated with a virus in a subject in need thereof, the method comprising administering to the subject in need thereof a pharmaceutical composition comprising: a therapeutically effective amount of a dual HDAC3 and HDAC8 targeting agent, or a pharmaceutically acceptable salt thereof; a therapeutically effective amount of an agent capable of sensitizing or potentiating induction of the lytic phase of the virus, or a pharmaceutically acceptable salt thereof; and a pharmaceutically acceptable excipient. In another aspect, the present disclosure provides a method of preventing a disease associated with a virus in a subject in need thereof, the method comprising administering to the subject in need thereof a pharmaceutical composition comprising: a therapeutically effective amount of a dual HDAC3 and HDAC8 targeting agent, or a pharmaceutically acceptable salt thereof; a therapeutically effective amount of an agent capable of sensitizing or potentiating induction of the lytic phase of the virus, or a pharmaceutically acceptable salt thereof; and a pharmaceutically acceptable excipient.

In some embodiments, the pharmaceutical composition is administered to the subject in need thereof after the subject in need thereof has been diagnosed as comprising malignant or pre-malignant cells infected with the virus. In some embodiments, the pharmaceutical composition is administered to the subject in need thereof after the subject in need thereof has been diagnosed as comprising malignant cells infected with the virus. In some embodiments, the pharmaceutical composition is administered to the subject in need thereof after the subject in need thereof has been diagnosed as comprising pre-malignant cells infected with the virus. In some embodiments, the virus is in the latent state or the abortive lytic state. In some embodiments, the virus is in the latent state. In some embodiments, the virus is in the abortive lytic state.

In another aspect, the present disclosure provides a dual HDAC3 and HDAC8 targeting agent, or a pharmaceutically acceptable salt thereof, for use in the treatment or prevention of a disease associated with a virus. In another aspect, the present disclosure provides a dual HDAC3 and HDAC8 targeting agent, or a pharmaceutically acceptable salt thereof, for use in the treatment of a disease associated with a virus. In another aspect, the present disclosure provides a dual HDAC3 and HDAC8 targeting agent, or a pharmaceutically acceptable salt thereof, for use in the prevention of a disease associated with a virus.

In another aspect, the present disclosure provides a dual HDAC3 and HDAC8 targeting agent, or a pharmaceutically acceptable salt thereof, for use in the manufacture of a medicament for the treatment or prevention of a disease associated with a virus. In another aspect, the present disclosure provides a dual HDAC3 and HDAC8 targeting agent, or a pharmaceutically acceptable salt thereof, for use in the manufacture of a medicament for the treatment of a disease associated with a virus. In another aspect, the present disclosure provides a dual HDAC3 and HDAC8 targeting agent, or a pharmaceutically acceptable salt thereof, for use in the manufacture of a medicament for the prevention of a disease associated with a virus.

In another aspect, the present disclosure provides a dual HDAC3 and HDAC8 targeting agent, or a pharmaceutically acceptable salt thereof; and an antiviral agent, or a pharmaceutically acceptable salt thereof, for use in the treatment or prevention of a disease associated with a virus. In another aspect, the present disclosure provides a dual HDAC3 and HDAC8 targeting agent, or a pharmaceutically acceptable salt thereof; and an antiviral agent, or a pharmaceutically acceptable salt thereof, for use in the treatment of a disease associated with a virus. In another aspect, the present disclosure provides a dual HDAC3 and HDAC8 targeting agent, or a pharmaceutically acceptable salt thereof; and an antiviral agent, or a pharmaceutically acceptable salt thereof, for use in the prevention of a disease associated with a virus.

In another aspect, the present disclosure provides a dual HDAC3 and HDAC8 targeting agent, or a pharmaceutically acceptable salt thereof; and an antiviral agent, or a pharmaceutically acceptable salt thereof, for use in the manufacture of a medicament for the treatment or prevention of a disease associated with a virus. In another aspect, the present disclosure provides a dual HDAC3 and HDAC8 targeting agent, or a pharmaceutically acceptable salt thereof; and an antiviral agent, or a pharmaceutically acceptable salt thereof, for use in the manufacture of a medicament for the treatment of a disease associated with a virus. In another aspect, the present disclosure provides a dual HDAC3 and HDAC8 targeting agent, or a pharmaceutically acceptable salt thereof; and an antiviral agent, or a pharmaceutically acceptable salt thereof, for use in the manufacture of a medicament for the prevention of a disease associated with a virus.

In another aspect, the present disclosure provides a pharmaceutical composition for use in the treatment or prevention of a disease associated with a virus, wherein the pharmaceutical composition comprises: a therapeutically effective amount of a dual HDAC3 and HDAC8 targeting agent, or a pharmaceutically acceptable salt thereof; and a pharmaceutically acceptable excipient. In another aspect, the present disclosure provides a pharmaceutical composition for use in the treatment of a disease associated with a virus, wherein the pharmaceutical composition comprises: a therapeutically effective amount of a dual HDAC3 and HDAC8 targeting agent, or a pharmaceutically acceptable salt thereof; and a pharmaceutically acceptable excipient. In another aspect, the present disclosure provides a pharmaceutical composition for use in the prevention of a disease associated with a virus, wherein the pharmaceutical composition comprises: a therapeutically effective amount of a dual HDAC3 and HDAC8 targeting agent, or a pharmaceutically acceptable salt thereof; and a pharmaceutically acceptable excipient.

In another aspect, the present disclosure provides a pharmaceutical composition for use in the manufacture of a medicament for the treatment or prevention of a disease associated with a virus, wherein the pharmaceutical composition comprises: a therapeutically effective amount of a dual HDAC3 and HDAC8 targeting agent, or a pharmaceutically acceptable salt thereof; and a pharmaceutically acceptable excipient. In another aspect, the present disclosure provides a pharmaceutical composition for use in the manufacture of a medicament for the treatment of a disease associated with a virus, wherein the pharmaceutical composition comprises: a therapeutically effective amount of a dual HDAC3 and HDAC8 targeting agent, or a pharmaceutically acceptable salt thereof; and a pharmaceutically acceptable excipient. In another aspect, the present disclosure provides a pharmaceutical composition for use in the manufacture of a medicament for the prevention of a disease associated with a virus, wherein the pharmaceutical composition comprises: a therapeutically effective amount of a dual HDAC3 and HDAC8 targeting agent, or a pharmaceutically acceptable salt thereof; and a pharmaceutically acceptable excipient.

In another aspect, the present disclosure provides a pharmaceutical composition for use in the treatment or prevention of a disease associated with a virus, wherein the pharmaceutical composition comprises: a therapeutically effective amount of a dual HDAC3 and HDAC8 targeting agent, or a pharmaceutically acceptable salt thereof; a therapeutically effective amount of an antiviral agent, or a pharmaceutically acceptable salt thereof; and a pharmaceutically acceptable excipient. In another aspect, the present disclosure provides a pharmaceutical composition for use in the treatment of a disease associated with a virus, wherein the pharmaceutical composition comprises: a therapeutically effective amount of a dual HDAC3 and HDAC8 targeting agent, or a pharmaceutically acceptable salt thereof; a therapeutically effective amount of an antiviral agent, or a pharmaceutically acceptable salt thereof; and a pharmaceutically acceptable excipient. In another aspect, the present disclosure provides a pharmaceutical composition for use in the prevention of a disease associated with a virus, wherein the pharmaceutical composition comprises: a therapeutically effective amount of a dual HDAC3 and HDAC8 targeting agent, or a pharmaceutically acceptable salt thereof; a therapeutically effective amount of an antiviral agent, or a pharmaceutically acceptable salt thereof; and a pharmaceutically acceptable excipient.

In another aspect, the present disclosure provides a pharmaceutical composition for use in the manufacture of a medicament for the treatment or prevention of a disease associated with a virus, wherein the pharmaceutical composition comprises: a therapeutically effective amount of a dual HDAC3 and HDAC8 targeting agent, or a pharmaceutically acceptable salt thereof; a therapeutically effective amount of an antiviral agent, or a pharmaceutically acceptable salt thereof; and a pharmaceutically acceptable excipient. In another aspect, the present disclosure provides a pharmaceutical composition for use in the manufacture of a medicament for the treatment of a disease associated with a virus, wherein the pharmaceutical composition comprises: a therapeutically effective amount of a dual HDAC3 and HDAC8 targeting agent, or a pharmaceutically acceptable salt thereof; a therapeutically effective amount of an antiviral agent, or a pharmaceutically acceptable salt thereof; and a pharmaceutically acceptable excipient. In another aspect, the present disclosure provides a pharmaceutical composition for use in the manufacture of a medicament for the prevention of a disease associated with a virus, wherein the pharmaceutical composition comprises: a therapeutically effective amount of a dual HDAC3 and HDAC8 targeting agent, or a pharmaceutically acceptable salt thereof; a therapeutically effective amount of an antiviral agent, or a pharmaceutically acceptable salt thereof; and a pharmaceutically acceptable excipient.

### Additional Embodiments of Methods of Treatment and Prevention

The present disclosure encompasses any and all molecules that are dual HDAC3 and HDAC8 targeting agents. In some embodiments, the dual HDAC3 and HDAC8 targeting agent binds to HDAC3 and HDAC8 without degrading HDAC3 and/or HDAC8. In some embodiments, the dual HDAC3 and HDAC8 targeting agent degrades HDAC3 and/or HDAC8. In some embodiments, the dual HDAC3 and HDAC8 targeting agent degrades HDAC3 and HDAC8. HDAC3 degraders are described in International Patent Publication No. WO 2021/236491.

YX968 is a heterobifunctional degrader (i.e., proteolysis targeting chimera (PROTAC)) that selectively and specifically targets and degrades HDAC3 and HDAC8. The effects of YX968 treatment on cultured cells have been characterized and described. See, e.g., Xiao Y, Hale S, Awasthee N, Meng C, Zhang X, Liu Y, Ding H, Huo Z, Lv D, Zhang W, He M, Zheng G, Liao D. HDAC3 and HDAC8 PROTAC dual degrader reveals roles of histone acetylation in gene regulation. Cell Chem Biol. 2023 Nov. 16;30(11):1421-1435.e12. doi: 10.1016/j.chembiol.2023.07.010. Epub 2023 Aug. 11. PMID: 37572669; PMCID: PMC10802846, which is incorporated herein by reference in its entirety.

Chromatin is DNA wrapped around histones. Heterochomatin, or compacted chromatin, is created and maintained by the application of post-translational modifications to histones. Important examples of post-translational modifications that lead to heterochromatin formation (also known as a silencing marks) are the trimethylation of lysine 27 of histone 3 (called H3K27me3) or trimethylation of lysine 9 of histone 3 (called H3K9me3). Opening or relaxation of chromatin is achieved through the application of different posttranslational modifications called activating marks. An example of an activating mark is the acetylation of lysine 27 of histone 3 (called H3K27ac).

Histone deacetylases (HDACs) are proteins that remove acetyl modifications, which are normally activating marks, from histones. An inhibitor of HDAC proteins (HDACi) prevents HDACs from removing acetyl marks, which allows them to accumulate on histones. This allows chromatin to relax and gene transcription to begin from previously silenced regions of the genome, whether it is a human genome or viral genome.

The EBV genome is maintained in a latent state in infected cells by the heterochromatinization of the EBV genome. Heavily heterochromatinized DNA is not normally transcribed. Without transcription of BZLF1, the lytic switch gene of EBV, EBV remains in the latent state. The abortive lytic state is characterized by expression of BZLF1 without expression of early (BMRF1, BMLF1, BGLF1, etc.) or late genes (BFRF3, BMRF2, BCRF1, etc.) or viral genome replication.

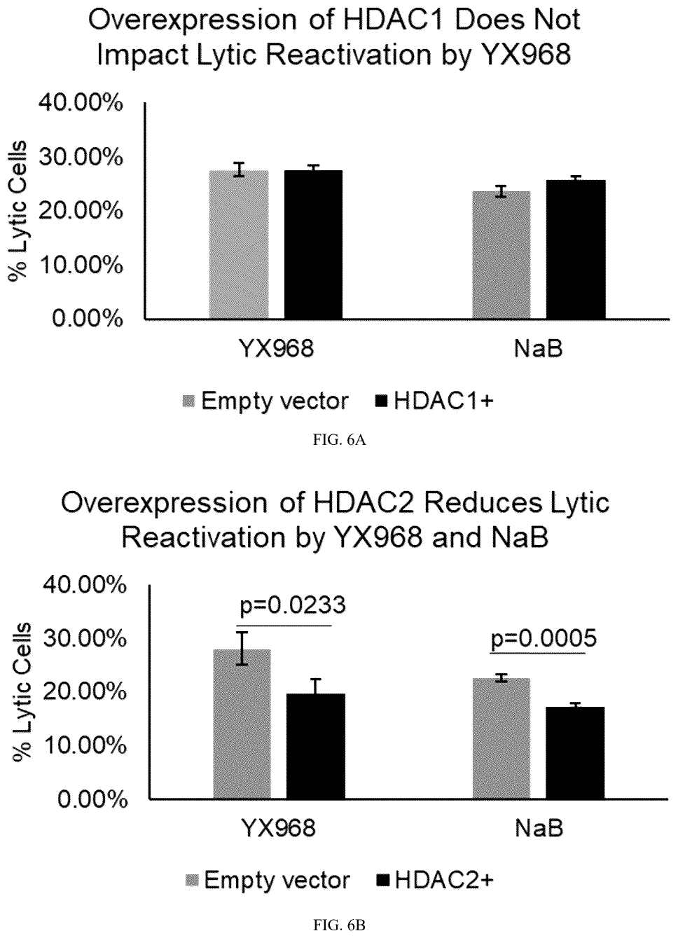

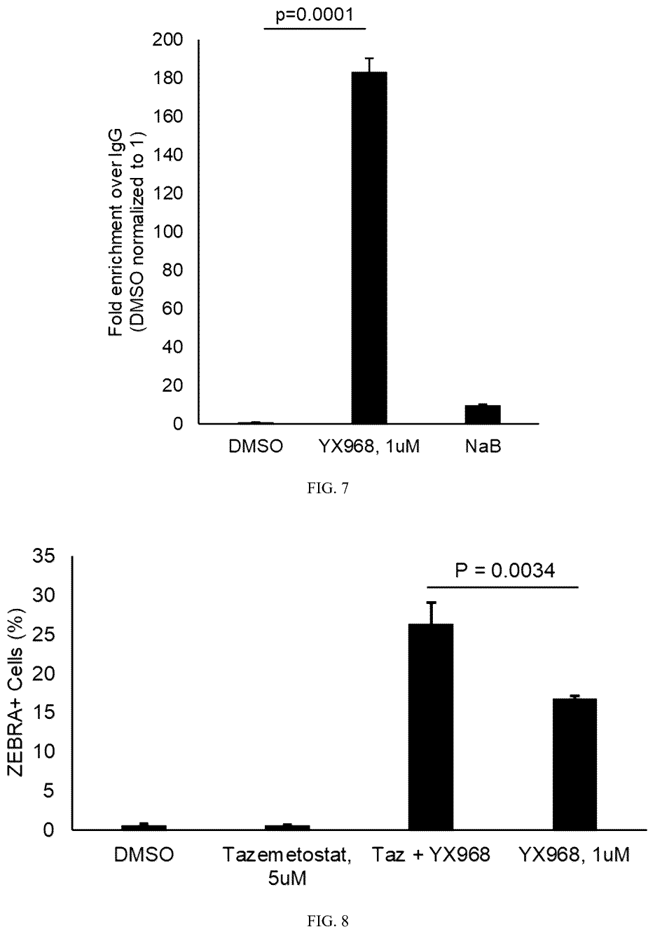

YX968 degrades HDAC3 and HDAC8, and at high concentrations, YX968 may also inhibit the functions of HDAC1 and HDAC2. Evidence of specific inhibition of HDAC2 can be found in FIG. 6B. The combined effect of HDAC degradation and inhibition causes an increase in the level of H3K27ac accumulation at the BZLF1 promoter (FIG. 7). This supports the derepression of BZLF1 and the upregulation of BZLF1 transcripts. BZLF1 upregulation begins the lytic phase of EBV.

Chemical modifications to YX968 that improve its stability, bioavailability, pharmacodynamics, pharmacokinetics, or any other characteristic that improve one or more attributes of YX968 for oncolytic therapy are within the scope of the present disclosure. Molecules that are not YX968, which may or may not be PROTACs, which also target HDAC3 and HDAC8, are understood to also be encompassed by the present disclosure.

In some embodiments, the dual HDAC3 and HDAC8 targeting agent is YX968, or a pharmaceutically acceptable salt thereof. In some embodiments, the dual HDAC3 and HDAC8 targeting agent is a pharmaceutically acceptable salt of YX968. In some embodiments, the dual HDAC3 and HDAC8 targeting agent is YX968.

In some embodiments, the dual HDAC3 and HDAC8 targeting agent is:

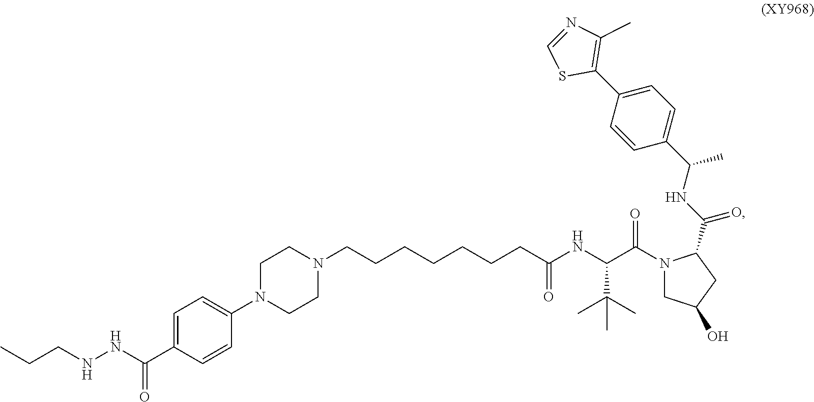 [SMILES: CCCNNC(=O)c1ccc(N2CCN(CCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

or a pharmaceutically acceptable salt thereof. In some embodiments, the dual HDAC3 and HDAC8 targeting agent is a pharmaceutically acceptable salt of:

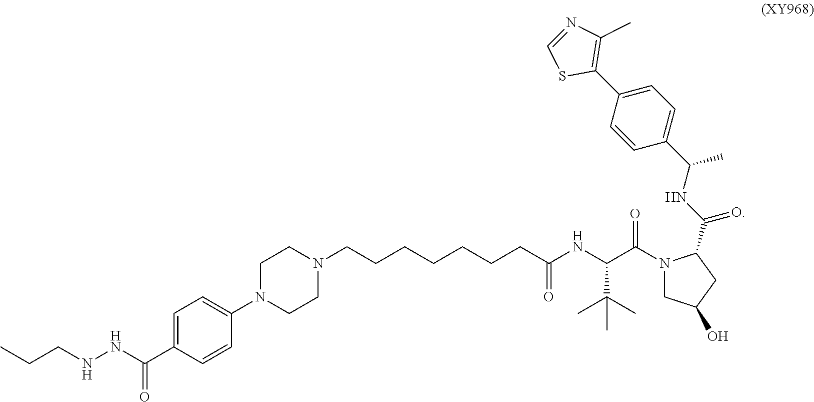 [SMILES: CCCNNC(=O)c1ccc(N2CCN(CCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

In some embodiments, the dual HDAC3 and HDAC8 targeting agent is:

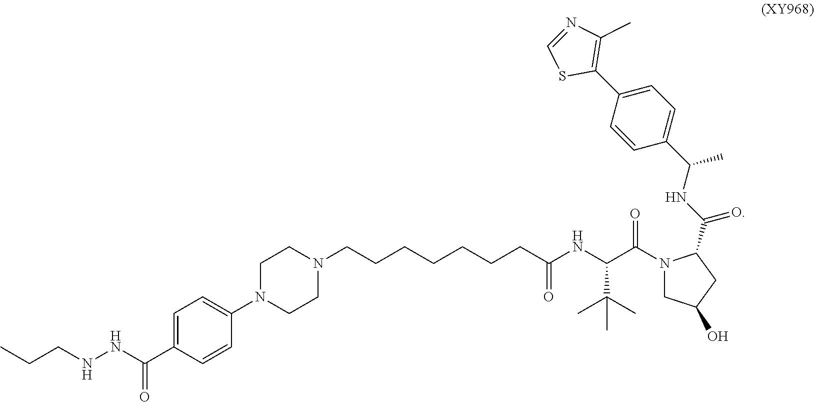 [SMILES: CCCNNC(=O)c1ccc(N2CCN(CCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

YX968 and other molecules that induce the lytic phase can potentially be combined into a pharmaceutical composition that contains molecules that inhibit EBV from creating infectious virions, such as ganciclovir, cidofovir, acyclovir, and foscarnet. New formulations of these compositions, or compositions that similarly incorporate into the cell and viral genome to block genome replication (i.e., are used for the same purpose) are within the scope of the present disclosure.

In some embodiments, the antiviral agent is ganciclovir, cidofovir, acyclovir, or foscarnet, or a pharmaceutically acceptable salt thereof. In some embodiments, the antiviral agent is a pharmaceutically acceptable salt of ganciclovir, cidofovir, acyclovir, or foscarnet. In some embodiments, the antiviral agent is ganciclovir, cidofovir, acyclovir, or foscarnet.

In some embodiments, the antiviral agent is ganciclovir, or a pharmaceutically acceptable salt thereof. In some embodiments, the antiviral agent is a pharmaceutically acceptable salt of ganciclovir. In some embodiments, the antiviral agent is ganciclovir.

In some embodiments, the antiviral agent is cidofovir, or a pharmaceutically acceptable salt thereof. In some embodiments, the antiviral agent is a pharmaceutically acceptable salt of cidofovir. In some embodiments, the antiviral agent is cidofovir.

In some embodiments, the antiviral agent is acyclovir, or a pharmaceutically acceptable salt thereof. In some embodiments, the antiviral agent is a pharmaceutically acceptable salt of acyclovir. In some embodiments, the antiviral agent is acyclovir.

In some embodiments, the antiviral agent is foscarnet, or a pharmaceutically acceptable salt thereof. In some embodiments, the antiviral agent is a pharmaceutically acceptable salt of foscarnet. In some embodiments, the antiviral agent is foscarnet.

In some embodiments, the dual HDAC3 and HDAC8 targeting agent is YX968, or a pharmaceutically acceptable salt thereof; and the antiviral agent is ganciclovir, cidofovir, acyclovir, or foscarnet, or a pharmaceutically acceptable salt thereof. In some embodiments, the dual HDAC3 and HDAC8 targeting agent is YX968, or a pharmaceutically acceptable salt thereof; and the antiviral agent is a pharmaceutically acceptable salt of ganciclovir, cidofovir, acyclovir, or foscarnet. In some embodiments, the dual HDAC3 and HDAC8 targeting agent is YX968, or a pharmaceutically acceptable salt thereof; and the antiviral agent is ganciclovir, cidofovir, acyclovir, or foscarnet.

In some embodiments, the dual HDAC3 and HDAC8 targeting agent is a pharmaceutically acceptable salt of YX968; and the antiviral agent is ganciclovir, cidofovir, acyclovir, or foscarnet, or a pharmaceutically acceptable salt thereof. In some embodiments, the dual HDAC3 and HDAC8 targeting agent is a pharmaceutically acceptable salt of YX968; and the antiviral agent is a pharmaceutically acceptable salt of ganciclovir, cidofovir, acyclovir, or foscarnet. In some embodiments, the dual HDAC3 and HDAC8 targeting agent is a pharmaceutically acceptable salt of YX968; and the antiviral agent is ganciclovir, cidofovir, acyclovir, or foscarnet.

In some embodiments, the dual HDAC3 and HDAC8 targeting agent is YX968; and the antiviral agent is ganciclovir, cidofovir, acyclovir, or foscarnet, or a pharmaceutically acceptable salt thereof. In some embodiments, the dual HDAC3 and HDAC8 targeting agent is YX968; and the antiviral agent is a pharmaceutically acceptable salt of ganciclovir, cidofovir, acyclovir, or foscarnet. In some embodiments, the dual HDAC3 and HDAC8 targeting agent is YX968; and the antiviral agent is ganciclovir, cidofovir, acyclovir, or foscarnet.

In some embodiments, the dual HDAC3 and HDAC8 targeting agent is YX968, or a pharmaceutically acceptable salt thereof; and the antiviral agent is ganciclovir, or a pharmaceutically acceptable salt thereof. In some embodiments, the dual HDAC3 and HDAC8 targeting agent is YX968, or a pharmaceutically acceptable salt thereof; and the antiviral agent is a pharmaceutically acceptable salt of ganciclovir. In some embodiments, the dual HDAC3 and HDAC8 targeting agent is YX968, or a pharmaceutically acceptable salt thereof; and the antiviral agent is ganciclovir.

In some embodiments, the dual HDAC3 and HDAC8 targeting agent is a pharmaceutically acceptable salt of YX968; and the antiviral agent is ganciclovir, or a pharmaceutically acceptable salt thereof. In some embodiments, the dual HDAC3 and HDAC8 targeting agent is a pharmaceutically acceptable salt of YX968; and the antiviral agent is a pharmaceutically acceptable salt of ganciclovir. In some embodiments, the dual HDAC3 and HDAC8 targeting agent is a pharmaceutically acceptable salt of YX968; and the antiviral agent is ganciclovir.

In some embodiments, the dual HDAC3 and HDAC8 targeting agent is YX968; and the antiviral agent is ganciclovir, or a pharmaceutically acceptable salt thereof. In some embodiments, the dual HDAC3 and HDAC8 targeting agent is YX968; and the antiviral agent is a pharmaceutically acceptable salt of ganciclovir. In some embodiments, the dual HDAC3 and HDAC8 targeting agent is YX968; and the antiviral agent is ganciclovir.

In some embodiments, the dual HDAC3 and HDAC8 targeting agent is YX968, or a pharmaceutically acceptable salt thereof; and the antiviral agent is cidofovir, or a pharmaceutically acceptable salt thereof. In some embodiments, the dual HDAC3 and HDAC8 targeting agent is YX968, or a pharmaceutically acceptable salt thereof; and the antiviral agent is a pharmaceutically acceptable salt of cidofovir. In some embodiments, the dual HDAC3 and HDAC8 targeting agent is YX968, or a pharmaceutically acceptable salt thereof; and the antiviral agent is cidofovir.

In some embodiments, the dual HDAC3 and HDAC8 targeting agent is a pharmaceutically acceptable salt of YX968; and the antiviral agent is cidofovir, or a pharmaceutically acceptable salt thereof. In some embodiments, the dual HDAC3 and HDAC8 targeting agent is a pharmaceutically acceptable salt of YX968; and the antiviral agent is a pharmaceutically acceptable salt of cidofovir. In some embodiments, the dual HDAC3 and HDAC8 targeting agent is a pharmaceutically acceptable salt of YX968; and the antiviral agent is cidofovir.

In some embodiments, the dual HDAC3 and HDAC8 targeting agent is YX968; and the antiviral agent is cidofovir, or a pharmaceutically acceptable salt thereof. In some embodiments, the dual HDAC3 and HDAC8 targeting agent is YX968; and the antiviral agent is a pharmaceutically acceptable salt of cidofovir. In some embodiments, the dual HDAC3 and HDAC8 targeting agent is YX968; and the antiviral agent is cidofovir.

In some embodiments, the dual HDAC3 and HDAC8 targeting agent is YX968, or a pharmaceutically acceptable salt thereof; and the antiviral agent is acyclovir, or a pharmaceutically acceptable salt thereof. In some embodiments, the dual HDAC3 and HDAC8 targeting agent is YX968, or a pharmaceutically acceptable salt thereof; and the antiviral agent is a pharmaceutically acceptable salt of acyclovir. In some embodiments, the dual HDAC3 and HDAC8 targeting agent is YX968, or a pharmaceutically acceptable salt thereof; and the antiviral agent is acyclovir.

In some embodiments, the dual HDAC3 and HDAC8 targeting agent is a pharmaceutically acceptable salt of YX968; and the antiviral agent is acyclovir, or a pharmaceutically acceptable salt thereof. In some embodiments, the dual HDAC3 and HDAC8 targeting agent is a pharmaceutically acceptable salt of YX968; and the antiviral agent is a pharmaceutically acceptable salt of acyclovir. In some embodiments, the dual HDAC3 and HDAC8 targeting agent is a pharmaceutically acceptable salt of YX968; and the antiviral agent is acyclovir.

In some embodiments, the dual HDAC3 and HDAC8 targeting agent is YX968; and the antiviral agent is acyclovir, or a pharmaceutically acceptable salt thereof. In some embodiments, the dual HDAC3 and HDAC8 targeting agent is YX968; and the antiviral agent is a pharmaceutically acceptable salt of acyclovir. In some embodiments, the dual HDAC3 and HDAC8 targeting agent is YX968; and the antiviral agent is acyclovir.

In some embodiments, the dual HDAC3 and HDAC8 targeting agent is YX968, or a pharmaceutically acceptable salt thereof; and the antiviral agent is foscarnet, or a pharmaceutically acceptable salt thereof. In some embodiments, the dual HDAC3 and HDAC8 targeting agent is YX968, or a pharmaceutically acceptable salt thereof; and the antiviral agent is a pharmaceutically acceptable salt of foscarnet. In some embodiments, the dual HDAC3 and HDAC8 targeting agent is YX968, or a pharmaceutically acceptable salt thereof; and the antiviral agent is foscarnet.

In some embodiments, the dual HDAC3 and HDAC8 targeting agent is a pharmaceutically acceptable salt of YX968; and the antiviral agent is foscarnet, or a pharmaceutically acceptable salt thereof. In some embodiments, the dual HDAC3 and HDAC8 targeting agent is a pharmaceutically acceptable salt of YX968; and the antiviral agent is a pharmaceutically acceptable salt of foscarnet. In some embodiments, the dual HDAC3 and HDAC8 targeting agent is a pharmaceutically acceptable salt of YX968; and the antiviral agent is foscarnet.

In some embodiments, the dual HDAC3 and HDAC8 targeting agent is YX968; and the antiviral agent is foscarnet, or a pharmaceutically acceptable salt thereof. In some embodiments, the dual HDAC3 and HDAC8 targeting agent is YX968; and the antiviral agent is a pharmaceutically acceptable salt of foscarnet. In some embodiments, the dual HDAC3 and HDAC8 targeting agent is YX968; and the antiviral agent is foscarnet.

The dual HDAC3 and HDAC8 targeting agent (e.g., YX968) can be combined with other HDAC inhibitors or any other molecule intended to induce the lytic phase of EBV. This combination can be in a single pharmaceutical composition, or as administration of the compounds separately. Such a combination may overcome the ability of EBV to resist lytic reactivation by subverting naturally occurring host mechanisms of gene silencing.

The dual HDAC3 and HDAC8 targeting agent (e.g., YX968) can be combined with any molecule which may sensitize or potentiate cells to the effects of lytic inducing agents. An example of a molecule that potentiates or sensitizes cells into transitioning into the lytic phase in response to a lytic inducing agent is tazemetostat (FIG. 8). Tazemetostat (also called EPZ-6438 and sold under the brand name Tazverik) is an EZH2 inhibitor. EZH2 is a member of the PRC2 (Polycomb Repressive Complex 2). This complex is known to transfer methyl groups to Histone H3 Lysine 27 which creates H3K27me3 marks. This is a known silencing mark that suppresses transcription at promoters in eukaryotic cells. The combination of a dual HDAC3 and HDAC8 targeting agent with an inhibitor of complexes of proteins or single proteins that introduce silencing marks on histones can be in a single pharmaceutical composition, or as administration of the compounds separately. Such a combination may more readily overcome the ability of EBV to resist lytic reactivation by subverting naturally occurring host mechanisms of gene silencing.

In some embodiments, the additional agent capable of sensitizing or potentiating induction of the lytic phase of the virus is an EZH2 inhibitor (e.g., tazemetostat). In some embodiments, the additional agent capable of sensitizing or potentiating induction of the lytic phase of the virus is tazemetostat, or a pharmaceutically acceptable salt thereof. In some embodiments, the additional agent capable of sensitizing or potentiating induction of the lytic phase of the virus is tazemetostat. In some embodiments, the additional agent capable of sensitizing or potentiating induction of the lytic phase of the virus is a pharmaceutically acceptable salt of tazemetostat.

In some embodiments, the dual HDAC3 and HDAC8 targeting agent is YX968, or a pharmaceutically acceptable salt thereof; and the additional agent capable of sensitizing or potentiating induction of the lytic phase of the virus is tazemetostat, or a pharmaceutically acceptable salt thereof. In some embodiments, the dual HDAC3 and HDAC8 targeting agent is YX968, or a pharmaceutically acceptable salt thereof; and the additional agent capable of sensitizing or potentiating induction of the lytic phase of the virus is a pharmaceutically acceptable salt of tazemetostat. In some embodiments, the dual HDAC3 and HDAC8 targeting agent is YX968, or a pharmaceutically acceptable salt thereof; and the additional agent capable of sensitizing or potentiating induction of the lytic phase of the virus is tazemetostat.

In some embodiments, the dual HDAC3 and HDAC8 targeting agent is a pharmaceutically acceptable salt of YX968; and the additional agent capable of sensitizing or potentiating induction of the lytic phase of the virus is tazemetostat, or a pharmaceutically acceptable salt thereof. In some embodiments, the dual HDAC3 and HDAC8 targeting agent is a pharmaceutically acceptable salt of YX968; and the additional agent capable of sensitizing or potentiating induction of the lytic phase of the virus is a pharmaceutically acceptable salt of tazemetostat. In some embodiments, the dual HDAC3 and HDAC8 targeting agent is a pharmaceutically acceptable salt of YX968; and the additional agent capable of sensitizing or potentiating induction of the lytic phase of the virus is tazemetostat.

In some embodiments, the dual HDAC3 and HDAC8 targeting agent is YX968; and the additional agent capable of sensitizing or potentiating induction of the lytic phase of the virus is tazemetostat, or a pharmaceutically acceptable salt thereof. In some embodiments, the dual HDAC3 and HDAC8 targeting agent is YX968; and the additional agent capable of sensitizing or potentiating induction of the lytic phase of the virus is a pharmaceutically acceptable salt of tazemetostat. In some embodiments, the dual HDAC3 and HDAC8 targeting agent is YX968; and the additional agent capable of sensitizing or potentiating induction of the lytic phase of the virus is tazemetostat.

In some embodiments, the method further comprises administering to the subject in need thereof a therapeutically effective amount of an additional agent capable of inducing the lytic phase of the virus, or a pharmaceutically acceptable salt thereof. In some embodiments, the pharmaceutical composition further comprises a therapeutically effective amount of an additional agent capable of inducing the lytic phase of the virus, or a pharmaceutically acceptable salt thereof.

In some embodiments, the additional agent capable of inducing the lytic phase of the virus is an HDAC inhibitor, a DNA methyltransferase inhibitor, a phorbol ester, a hypoxia-mimicking agent, or an immunoglobulin. In some embodiments, the additional agent capable of inducing the lytic phase of the virus is an HDAC inhibitor. In some embodiments, the additional agent capable of inducing the lytic phase of the virus is a DNA methyltransferase inhibitor. In some embodiments, the additional agent capable of inducing the lytic phase of the virus is a phorbol ester. In some embodiments, the additional agent capable of inducing the lytic phase of the virus is a hypoxia-mimicking agent. In some embodiments, the additional agent capable of inducing the lytic phase of the virus is an immunoglobulin.

In some embodiments, the additional agent capable of inducing the lytic phase of the virus is sodium butyrate, valproic acid, romidepsin, MS-275, apicidin, oxamflatin, Scriptaid, panobinostat, or nanatinostat, or a pharmaceutically acceptable salt thereof. In some embodiments, the additional agent capable of inducing the lytic phase of the virus is sodium butyrate, or a pharmaceutically acceptable salt thereof. In some embodiments, the additional agent capable of inducing the lytic phase of the virus is valproic acid, or a pharmaceutically acceptable salt thereof. In some embodiments, the additional agent capable of inducing the lytic phase of the virus is romidepsin, or a pharmaceutically acceptable salt thereof. In some embodiments, the additional agent capable of inducing the lytic phase of the virus is MS-275, or a pharmaceutically acceptable salt thereof. In some embodiments, the additional agent capable of inducing the lytic phase of the virus is apicidin, or a pharmaceutically acceptable salt thereof. In some embodiments, the additional agent capable of inducing the lytic phase of the virus is oxamflatin, or a pharmaceutically acceptable salt thereof. In some embodiments, the additional agent capable of inducing the lytic phase of the virus is Scriptaid, or a pharmaceutically acceptable salt thereof. In some embodiments, the additional agent capable of inducing the lytic phase of the virus is Panobinostat, or a pharmaceutically acceptable salt thereof. In some embodiments, the additional agent capable of inducing the lytic phase of the virus is nanatinostat, or a pharmaceutically acceptable salt thereof.

In some embodiments, the additional agent capable of inducing the lytic phase of the virus is sodium butyrate, valproic acid, romidepsin, MS-275, apicidin, oxamflatin, Scriptaid, panobinostat, or nanatinostat. In some embodiments, the additional agent capable of inducing the lytic phase of the virus is sodium butyrate. In some embodiments, the additional agent capable of inducing the lytic phase of the virus is valproic acid. In some embodiments, the additional agent capable of inducing the lytic phase of the virus is romidepsin. In some embodiments, the additional agent capable of inducing the lytic phase of the virus is MS-275. In some embodiments, the additional agent capable of inducing the lytic phase of the virus is apicidin. In some embodiments, the additional agent capable of inducing the lytic phase of the virus is oxamflatin. In some embodiments, the additional agent capable of inducing the lytic phase of the virus is Scriptaid. In some embodiments, the additional agent capable of inducing the lytic phase of the virus is panobinostat. In some embodiments, the additional agent capable of inducing the lytic phase of the virus is nanatinostat.

In some embodiments, the virus is a herpesvirus. In some embodiments, the virus is a gamma-herpesvirus. In some embodiments, the virus is Epstein-Barr virus (EBV) or Kaposi's sarcoma-associated herpesvirus (KSHV). In some embodiments, the virus is Epstein-Barr virus (EBV). In some embodiments, the virus is Kaposi's sarcoma-associated herpesvirus (KSHV).

In some embodiments, the virus is in the latent state or the abortive lytic state. In some embodiments, the virus is in the latent state. In some embodiments, the virus is in the abortive lytic state.

EBV cancers that can be treated with oncolytic therapies include, but are not limited to, iatrogenic lymphoproliferative disorder/lymphomas, post-transplant lymphoproliferative disorder (PTLD)/lymphomas, Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cell carcinoma, AIDS-related lymphomas, chronic active EBV infection (CAEBV), and EBV-positive gastric carcinomas.

In some embodiments, the disease is a proliferative disease. In some embodiments, the proliferative disease is a lymphoproliferative disease. In some embodiments, the lymphoproliferative disease is iatrogenic lymphoproliferative disorder, iatrogenic lymphoma, post-transplant lymphoproliferative disorder (PTLD), post-transplant lymphoma, Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cell carcinoma, AIDS-related lymphoma, chronic active EBV infection (CAEBV), hemophagocytic lymphohistiocytosis, or EBV-positive gastric carcinoma.

In some embodiments, the lymphoproliferative disease is iatrogenic lymphoproliferative disorder. In some embodiments, the lymphoproliferative disease is iatrogenic lymphoma. In some embodiments, the lymphoproliferative disease is post-transplant lymphoproliferative disorder (PTLD). In some embodiments, the lymphoproliferative disease is post-transplant lymphoma. In some embodiments, the lymphoproliferative disease is Burkitt lymphoma. In some embodiments, the lymphoproliferative disease is B-cell non-Hodgkin lymphoma. In some embodiments, the lymphoproliferative disease is diffuse large B-cell lymphoma (DLBCL). In some embodiments, the lymphoproliferative disease is CNS lymphoma. In some embodiments, the lymphoproliferative disease is NK/T-cell lymphoma. In some embodiments, the lymphoproliferative disease is T cell non-Hodgkin lymphoma. In some embodiments, the lymphoproliferative disease is Hodgkin lymphoma. In some embodiments, the lymphoproliferative disease is nasopharyngeal cell carcinoma. In some embodiments, the lymphoproliferative disease is AIDS-related lymphoma. In some embodiments, the lymphoproliferative disease is chronic active EBV infection (CAEBV). In some embodiments, the lymphoproliferative disease is hemophagocytic lymphohistiocytosis. In some embodiments, the lymphoproliferative disease is EBV-positive gastric carcinoma.

In some embodiments, the proliferative disease is a cancer. In some embodiments, the cancer is a lymphoid malignancy. In some embodiments, the cancer is iatrogenic lymphoma, post-transplant lymphoma, Burkitt lymphoma, B-cell non-Hodgkin lymphoma, diffuse large B-cell lymphoma (DLBCL), central nervous system (CNS) lymphoma, NK/T-cell lymphoma, T cell non-Hodgkin lymphoma, or Hodgkin's lymphoma.

In some embodiments, the cancer is iatrogenic lymphoma. In some embodiments, the cancer is post-transplant lymphoma. In some embodiments, the cancer is Burkitt lymphoma. In some embodiments, the cancer is B-cell non-Hodgkin lymphoma. In some embodiments, the cancer is diffuse large B-cell lymphoma (DLBCL). In some embodiments, the cancer is central nervous system (CNS) lymphoma. In some embodiments, the cancer is NK/T-cell lymphoma. In some embodiments, the cancer is T cell non-Hodgkin lymphoma. In some embodiments, the cancer is Hodgkin's lymphoma.

In some embodiments, the cancer is a non-lymphoid malignancy. In some embodiments, the cancer is EBV-positive gastric carcinoma or nasopharyngeal cell carcinoma. In some embodiments, the cancer is EBV-positive gastric carcinoma. In some embodiments, the cancer is EBV-positive nasopharyngeal cell carcinoma.

In some embodiments, the cancer is associated with human immunodeficiency virus. In some embodiments, the cancer is oral hairy leukoplakia, central nervous system (CNS) lymphoma, or AIDS-related lymphoma. In some embodiments, the cancer is oral hairy leukoplakia. In some embodiments, the cancer is central nervous system (CNS) lymphoma. In some embodiments, the cancer is AIDS-related lymphoma.

In some embodiments, the disease is chronic active EBV infection (CAEBV).

Persons with multiple sclerosis (MS) may also benefit from the killing of their EBV positive B cells, as MS has been causally linked to EBV.

In some embodiments, the disease is multiple sclerosis (MS).

In some embodiments, the disease is associated with Kaposi's sarcoma-associated herpesvirus (KSHV).

In some embodiments, the disease is a primary effusion lymphoma (PEL), multicentric Castleman's disease, or Kaposi's sarcoma. In some embodiments, the disease is a primary effusion lymphoma (PEL). In some embodiments, the disease is multicentric Castleman's disease. In some embodiments, the disease is Kaposi's sarcoma.

In some embodiments, the subject in need thereof has not been administered one or more additional pharmaceutical agents and/or radiation. In some embodiments, the subject in need thereof has not been administered one or more additional pharmaceutical agents and radiation. In some embodiments, the subject in need thereof has not been administered one or more additional pharmaceutical agents or radiation. In some embodiments, the subject in need thereof has not been administered one or more additional pharmaceutical agents. In some embodiments, the subject in need thereof has not been administered radiation.

In some embodiments, the subject in need thereof has been administered one or more additional pharmaceutical agents and/or radiation. In some embodiments, the subject in need thereof has been administered one or more additional pharmaceutical agents and radiation. In some embodiments, the subject in need thereof has been administered one or more additional pharmaceutical agents or radiation. In some embodiments, the subject in need thereof has been administered one or more additional pharmaceutical agents. In some embodiments, the subject in need thereof has been administered radiation.

In some embodiments, the method further comprises administering to the subject in need thereof one or more additional pharmaceutical agents and/or radiation. In some embodiments, the method further comprises administering to the subject in need thereof one or more additional pharmaceutical agents and radiation. In some embodiments, the method further comprises administering to the subject in need thereof one or more additional pharmaceutical agents or radiation. In some embodiments, the method further comprises administering to the subject in need thereof one or more additional pharmaceutical agents. In some embodiments, the method further comprises administering to the subject in need thereof radiation.

In some embodiments, the one or more additional pharmaceutical agents are chemotherapeutic agents and/or PARP inhibitors. In some embodiments, the one or more additional pharmaceutical agents are chemotherapeutic agents and PARP inhibitors. In some embodiments, the one or more additional pharmaceutical agents are chemotherapeutic agents or PARP inhibitors. In some embodiments, the one or more additional pharmaceutical agents are chemotherapeutic agents. In some embodiments, the one or more additional pharmaceutical agents are PARP inhibitors. In some embodiments, the one or more additional pharmaceutical agents are CODOX-M/IVAC.

An EBV-positive LPD or EBV-associated malignancy or pre-malignancy in an immunosuppressed or non-immunosuppressed subject can be treated according to methods described herein. Cancer cells that can be killed or their proliferation inhibited include, but are not limited to, iatrogenic lymphoproliferative disorder/lymphomas, post-transplant lymphoproliferative disorder (PTLD)/lymphomas, Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cell carcinoma, AIDS-related ymphomas, chronic active EBV infection (CAEBV), and EBV-positive gastric carcinomas.

In some embodiments, the subject in need thereof is non-immunosuppressed. In some embodiments, the subject in need thereof has not been administered an immunosuppressive or immunomodulatory agent.

In some embodiments, the subject in need thereof is immunosuppressed. In some embodiments, the subject in need thereof has been administered an immunosuppressive or immunomodulatory agent.

In some embodiments, the method further comprises administering to the subject in need thereof an immunosuppressive or immunomodulatory agent. In some embodiments, the immunosuppressive or immunomodulatory agent is selected from corticosteroids (e.g., prednisone (Deltasone, Orasone), budesonide (Entocort EC), prednisolone (Millipred)), Janus kinase inhibitors (e.g., tofacitinib (Xeljanz)), calcineurin inhibitors (e.g., cyclosporine (Neoral, Sandimmune, SangCya), tacrolimus (Astagraf XL, Envarsus XR, Prograf)), mTOR inhibitors (e.g., sirolimus (Rapamune), everolimus (Afinitor, Zortress), inosine monophosphate dehydrogenase (IMDH) inhibitors (e.g., azathioprine (Azasan, Imuran), leflunomide (Arava), mycophenolate (CellCept, Myfortic), biologics (e.g., antibody, monoclonal antibody) (e.g., abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta)), and antimetabolites (e.g., methotrexate).

In some embodiments, the immunosuppressive or immunomodulatory agent is a corticosteroid (e.g., prednisone (Deltasone, Orasone), budesonide (Entocort EC), prednisolone (Millipred)). In some embodiments, the corticosteroid is prednisone (Deltasone, Orasone). In some embodiments, the corticosteroid is budesonide (Entocort EC). In some embodiments, the corticosteroid is prednisolone (Millipred). In some embodiments, the immunosuppressive or immunomodulatory agent is a Janus kinase inhibitor (e.g., tofacitinib (Xeljanz)). In some embodiments, the Janus kinase inhibitor is tofacitinib (Xeljanz). In some embodiments, the immunosuppressive or immunomodulatory agent is a calcineurin inhibitor (e.g., cyclosporine (Neoral, Sandimmune, SangCya). In some embodiments, the calcineurin inhibitor is cyclosporine (Neoral, Sandimmune, SangCya). In some embodiments, the immunosuppressive or immunomodulatory agent is tacrolimus (Astagraf XL, Envarsus XR, Prograf)). In some embodiments, the immunosuppressive or immunomodulatory agent is an mTOR inhibitor (e.g., sirolimus (Rapamune), everolimus (Afinitor, Zortress). In some embodiments, the mTOR inhibitor is sirolimus (Rapamune). In some embodiments, the mTOR inhibitor is everolimus (Afinitor, Zortress). In some embodiments, the immunosuppressive or immunomodulatory agent is an inosine monophosphate dehydrogenase (IMDH) inhibitor (e.g., azathioprine (Azasan, Imuran), leflunomide (Arava), mycophenolate (CellCept, Myfortic). In some embodiments, the IMDH inhibitor is azathioprine (Azasan, Imuran). In some embodiments, the IMDH inhibitor is leflunomide (Arava). In some embodiments, the IMDH inhibitor is mycophenolate (CellCept, Myfortic). In some embodiments, the immunosuppressive or immunomodulatory agent is a biologic (e.g., abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta)). In some embodiments, the biologic is abatacept (Orencia). In some embodiments, the biologic is adalimumab (Humira). In some embodiments, the biologic is anakinra (Kineret). In some embodiments, the biologic is certolizumab (Cimzia). In some embodiments, the biologic is etanercept (Enbrel). In some embodiments, the biologic is golimumab (Simponi). In some embodiments, the biologic is infliximab (Remicade). In some embodiments, the biologic is ixekizumab (Taltz). In some embodiments, the biologic is natalizumab (Tysabri). In some embodiments, the biologic is rituximab (Rituxan). In some embodiments, the biologic is secukinumab (Cosentyx). In some embodiments, the biologic is tocilizumab (Actemra). In some embodiments, the biologic is ustekinumab (Stelara). In some embodiments, the biologic is vedolizumab (Entyvio). In some embodiments, the biologic is basiliximab (Simulect). In some embodiments, the biologic is daclizumab (Zinbryta). In some embodiments, the immunosuppressive or immunomodulatory agent is an antimetabolite (e.g., methotrexate). In some embodiments, the antimetabolite is methotrexate.

In some embodiments, the method prevents or reduces the risk of developing EBV+ LPD in a subject that has been on or is currently on T cell suppressive therapy or anti-TNF alpha therapy. The T cell suppressive therapy can be, but is not limited to, antithymocyte globulin or recombinant antithymocyte globulin therapy.

In some embodiments, the subject has been administered one or more of T cell suppressive therapy and anti-TNF alpha therapy.

In some embodiments, the T cell suppressive therapy is antithymocyte globulin or recombinant antithymocyte globulin therapy. In some embodiments, the T cell suppressive therapy is antithymocyte globulin therapy. In some embodiments, the T cell suppressive therapy is recombinant antithymocyte globulin therapy.

In some embodiments, the subject in need thereof has a lymphoproliferative disease. In some embodiments, the subject in need thereof has a lymphoproliferative disease (LPD). In some embodiments, the subject in need thereof has an EBV-positive lymphoproliferative disease (LPD). In some embodiments, the subject in need thereof has an KSHV-positive lymphoproliferative disease (LPD).

In some embodiments, the method prevents the subject in need thereof from developing a lymphoproliferative disease (LPD). In some embodiments, the method prevents the subject in need thereof from developing an EBV-positive lymphoproliferative disease (LPD). In some embodiments, the method prevents the subject in need thereof from developing an KSHV-positive lymphoproliferative disease (LPD).

In some embodiments, the method prevents the subject in need thereof from developing an EBV-positive lymphoproliferative disease (LPD), malignancy, or pre-malignancy, wherein the subject in need thereof is a transplant recipient. Lymphoproliferative diseases that can be prevented or for which the risk can be reduced include, but are not limited to, iatrogenic lymphoproliferative disorder/lymphomas, post-transplant lymphoproliferative disorder (PTLD)/lymphomas, Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cell carcinoma, AIDS-related lymphomas, chronic active EBV infection (CAEBV), and EBV-positive gastric carcinomas.

In some embodiments, the subject in need thereof is a transplant recipient. In some embodiments, the transplant is a solid organ transplant or a hematopoietic transplant. In some embodiments, the transplant is a solid organ transplant. In some embodiments, the transplant is a hematopoietic transplant.

In some embodiments, the methods can be used to treat persons who have lymphoproliferative diseases despite evidence of elimination of the malignant cells. In some embodiments the therapeutic value will be in halting the continued expansion of proliferative cells rather than eliminating them completely.

In some embodiments, the method comprises continuing administration until the subject in need thereof is in remission from a cancer. In some embodiments, the cancer is iatrogenic lymphoma, post-transplant lymphoma, Burkitt lymphoma, B-cell non-Hodgkin lymphoma, diffuse large B-cell lymphoma (DLBCL), central nervous system (CNS) lymphoma, NK/T-cell lymphoma, T cell non-Hodgkin lymphoma, or Hodgkin's lymphoma. In some embodiments, the cancer is EBV-positive gastric carcinoma or nasopharyngeal cell carcinoma. In some embodiments, the cancer is oral hairy leukoplakia, central nervous system (CNS) lymphoma, or AIDS-related lymphoma.

In some embodiments, the subject in need thereof comprises EBV-positive cells. In some embodiments, the subject in need thereof comprises one or more of EBV-positive cancer cells and EBV-positive pre-cancerous cells. In some embodiments, the subject in need thereof comprises EBV-positive cancer cells and EBV-positive pre-cancerous cells. In some embodiments, the subject in need thereof comprises EBV-positive cancer cells. In some embodiments, the subject in need thereof comprises EBV-positive pre-cancerous cells.

In some embodiments, the EBV is in a latent lytic state or abortive lytic state. In some embodiments, the EBV is in a latent lytic state. In some embodiments, the EBV is in an abortive lytic state. In some embodiments, the method comprises inducing a conversion of EBV from an abortive lytic state to a lytic state. In some embodiments, the method comprises inducing a conversion of EBV from a latent state to a lytic state.

In some embodiments, the method comprises administering the dual HDAC3 and HDAC8 targeting agent, or the pharmaceutical composition comprising the dual HDAC3 and HDAC8 targeting agent, at the same time as the presence of EBV+ cells with EBV in the latent or abortive lytic state is established. In some embodiments, the method comprises administering the dual HDAC3 and HDAC8 targeting agent, or the pharmaceutical composition comprising the dual HDAC3 and HDAC8 targeting agent, after the presence of EBV+ cells with EBV in the latent or abortive lytic state has been established. In some embodiments, the method comprises administering the dual HDAC3 and HDAC8 targeting agent, or the pharmaceutical composition comprising the dual HDAC3 and HDAC8 targeting agent, until the EBV+ cells with EBV in the latent or abortive lytic state have been eliminated.

In some embodiments, EBV levels in the subject in need thereof are monitored, and the pharmaceutical composition containing a dual HDAC3 and HDAC8 targeting agent (such as YX968) is administered to the subject in need thereof if the levels of EBV in the subject increase.

In some embodiments, the method further comprises determining an initial level of EBV in the subject in need thereof; and determining at a later time a subsequent level of EBV in the subject in need thereof; wherein the administration occurs when the subsequent level of EBV is greater than the initial level of EBV.

In some embodiments, the subject in need thereof comprises KSHV-positive cells. In some embodiments, the subject in need thereof comprises one or more of KSHV-positive cancer cells and KSHV-positive pre-cancerous cells. In some embodiments, the subject in need thereof comprises KSHV-positive cancer cells and KSHV-positive pre-cancerous cells. In some embodiments, the subject in need thereof comprises KSHV-positive cancer cells. In some embodiments, the subject in need thereof comprises KSHV-positive pre-cancerous cells.

In some embodiments, the KSHV is in a latent lytic state or abortive lytic state. In some embodiments, the KSHV is in a latent lytic state. In some embodiments, the KSHV is in an abortive lytic state. In some embodiments, the method comprises inducing a conversion of KSHV from an abortive lytic state to a lytic state. In some embodiments, the method comprises inducing a conversion of KSHV from a latent state to a lytic state.

In some embodiments, the method comprises administering the dual HDAC3 and HDAC8 targeting agent, or the pharmaceutical composition comprising the dual HDAC3 and HDAC8 targeting agent, at the same time as the presence of KSHV+ cells with KSHV in the latent or abortive lytic state is established. In some embodiments, the method comprises administering the dual HDAC3 and HDAC8 targeting agent, or the pharmaceutical composition comprising the dual HDAC3 and HDAC8 targeting agent, after the presence of KSHV+ cells with KSHV in the latent or abortive lytic state has been established. In some embodiments, the method comprises administering the dual HDAC3 and HDAC8 targeting agent, or the pharmaceutical composition comprising the dual HDAC3 and HDAC8 targeting agent, until the KSHV+ cells with KSHV in the latent or abortive lytic state have been eliminated.

In some embodiments, KSHV levels in the subject in need thereof are monitored, and the pharmaceutical composition containing a dual HDAC3 and HDAC8 targeting agent (such as YX968) is administered to the subject in need thereof if the levels of KSHV in the subject increase.

In some embodiments, the method further comprises determining an initial level of KSHV in the subject in need thereof; and determining at a later time a subsequent level of KSHV in the subject in need thereof; wherein the administration occurs when the subsequent level of KSHV is greater than the initial level of KSHV.

In some embodiments, the subject in need thereof has been identified as being in need of treatment of the disease associated with the virus.

In some embodiments, the subject in need thereof has been identified as being in need of treatment of the disease associated with the virus by biopsy (e.g., bone marrow biopsy). In some embodiments, the subject in need thereof has been identified as being in need of treatment of the disease associated with the virus by biopsy (e.g., bone marrow biopsy) and staining for virus-specific markers (e.g., EBV-specific or KSHV-specific markers). In some embodiments, the subject in need thereof has been identified as being in need of treatment of the disease associated with the virus by blood test. In some embodiments, the subject in need thereof has been identified as being in need of treatment of the disease associated with the virus by blood test, wherein the blood test comprises determination that the subject in need thereof comprises B cells infected with the virus (e.g., EBV or KSHV).

In some embodiments, the subject in need thereof is diagnosed as comprising malignant or pre-malignant cells infected with the virus by biopsy (e.g., bone marrow biopsy). In some embodiments, the subject in need thereof is diagnosed as comprising malignant or pre-malignant cells infected with the virus by biopsy (e.g., bone marrow biopsy) and staining for virus-specific markers (e.g., EBV-specific or KSHV-specific markers). In some embodiments, the subject in need thereof is diagnosed as comprising malignant or pre-malignant cells infected with the virus by blood test. In some embodiments, the subject in need thereof is diagnosed as comprising malignant or pre-malignant cells infected with the virus by blood test, wherein the blood test comprises determination that the subject in need thereof comprises B cells infected with the virus (e.g., EBV or KSHV).

In some embodiments, the dual HDAC3 and HDAC8 targeting agent, or pharmaceutically acceptable salt thereof, degrades HDAC3. In some embodiments, the dual HDAC3 and HDAC8 targeting agent, or pharmaceutically acceptable salt thereof, degrades HDAC8. In some embodiments, the dual HDAC3 and HDAC8 targeting agent, or pharmaceutically acceptable salt thereof, inhibits HDAC2.

In some embodiments, the method comprises increasing an activating histone mark at the BZLF1 promoter. In some embodiments, the activating histone mark is H3K27Ac.

### Methods of Inhibiting Cell Proliferation or Promoting Apoptosis, Inducing a Lytic Phase of a Virus, and Reactivating a Virus

In another aspect, the present disclosure provides a method of inhibiting cell proliferation or promoting apoptosis in a subject in need thereof, the method comprising administering to the subject in need thereof an effective amount of a dual HDAC3 and HDAC8 targeting agent, or a pharmaceutically acceptable salt thereof.

In another aspect, the present disclosure provides a method of inducing a lytic phase of a virus in a subject in need thereof, the method comprising administering to the subject in need thereof an effective amount of a dual HDAC3 and HDAC8 targeting agent, or a pharmaceutically acceptable salt thereof.

In another aspect, the present disclosure provides a method of reactivating a virus in a subject in need thereof, the method comprising administering to the subject in need thereof an effective amount of a dual HDAC3 and HDAC8 targeting agent, or a pharmaceutically acceptable salt thereof.

In some embodiments, the method further comprises administering to the subject in need thereof an effective amount of an antiviral agent, or a pharmaceutically acceptable salt thereof.

In some embodiments, the method further comprises administering to the subject in need thereof an effective amount of an agent capable of sensitizing or potentiating induction of the lytic phase of the virus, or a pharmaceutically acceptable salt thereof.

In another aspect, the present disclosure provides a method of inhibiting cell proliferation or promoting apoptosis in a subject in need thereof, the method comprising administering to the subject in need thereof a pharmaceutical composition comprising: an effective amount of a dual HDAC3 and HDAC8 targeting agent, or a pharmaceutically acceptable salt thereof; and a pharmaceutically acceptable excipient.

In another aspect, the present disclosure provides a method of inducing a lytic phase of a virus in a subject in need thereof, the method comprising administering to the subject in need thereof a pharmaceutical composition comprising: an effective amount of a dual HDAC3 and HDAC8 targeting agent, or a pharmaceutically acceptable salt thereof; and a pharmaceutically acceptable excipient.

In another aspect, the present disclosure provides a method of reactivating a virus in a subject in need thereof, the method comprising administering to the subject in need thereof a pharmaceutical composition comprising: an effective amount of a dual HDAC3 and HDAC8 targeting agent, or a pharmaceutically acceptable salt thereof; and a pharmaceutically acceptable excipient.

In another aspect, the present disclosure provides a dual HDAC3 and HDAC8 targeting agent, or a pharmaceutically acceptable salt thereof, for use in the inhibition of cell proliferation or promoting apoptosis. In another aspect, the present disclosure provides a dual HDAC3 and HDAC8 targeting agent, or a pharmaceutically acceptable salt thereof, for use in the manufacture of a medicament for the inhibition of cell proliferation or promoting apoptosis.

In another aspect, the present disclosure provides a dual HDAC3 and HDAC8 targeting agent, or a pharmaceutically acceptable salt thereof, for use in the induction of a lytic phase of a virus. In another aspect, the present disclosure provides a dual HDAC3 and HDAC8 targeting agent, or a pharmaceutically acceptable salt thereof, for use in the manufacture of a medicament for the induction of a lytic phase of a virus.

In another aspect, the present disclosure provides a dual HDAC3 and HDAC8 targeting agent, or a pharmaceutically acceptable salt thereof, for use in the reactivation of a virus. In another aspect, the present disclosure provides a dual HDAC3 and HDAC8 targeting agent, or a pharmaceutically acceptable salt thereof, for use in the manufacture of a medicament for the reactivation of a virus.

In another aspect, the present disclosure provides a dual HDAC3 and HDAC8 targeting agent, or a pharmaceutically acceptable salt thereof; and an antiviral agent, or a pharmaceutically acceptable salt thereof, for use in the inhibition of cell proliferation or promoting apoptosis. In another aspect, the present disclosure provides a dual HDAC3 and HDAC8 targeting agent, or a pharmaceutically acceptable salt thereof; and an antiviral agent, or a pharmaceutically acceptable salt thereof, for use in the manufacture of a medicament for the inhibition of cell proliferation or promoting apoptosis.

In another aspect, the present disclosure provides a dual HDAC3 and HDAC8 targeting agent, or a pharmaceutically acceptable salt thereof; and an antiviral agent, or a pharmaceutically acceptable salt thereof, for use in the induction of a lytic phase of a virus. In another aspect, the present disclosure provides a dual HDAC3 and HDAC8 targeting agent, or a pharmaceutically acceptable salt thereof; and an antiviral agent, or a pharmaceutically acceptable salt thereof, for use in the manufacture of a medicament for the induction of a lytic phase of a virus.

In another aspect, the present disclosure provides a dual HDAC3 and HDAC8 targeting agent, or a pharmaceutically acceptable salt thereof; and an antiviral agent, or a pharmaceutically acceptable salt thereof, for use in the reactivation of a virus. In another aspect, the present disclosure provides a dual HDAC3 and HDAC8 targeting agent, or a pharmaceutically acceptable salt thereof; and an antiviral agent, or a pharmaceutically acceptable salt thereof, for use in the manufacture of a medicament for the reactivation of a virus.

In another aspect, the present disclosure provides a dual HDAC3 and HDAC8 targeting agent, or a pharmaceutically acceptable salt thereof; and an agent capable of sensitizing or potentiating induction of the lytic phase of the virus, or a pharmaceutically acceptable salt thereof, for use in the inhibition of cell proliferation or promoting apoptosis. In another aspect, the present disclosure provides a dual HDAC3 and HDAC8 targeting agent, or a pharmaceutically acceptable salt thereof; and an agent capable of sensitizing or potentiating induction of the lytic phase of the virus, or a pharmaceutically acceptable salt thereof, for use in the manufacture of a medicament for the inhibition of cell proliferation or promoting apoptosis.

In another aspect, the present disclosure provides a dual HDAC3 and HDAC8 targeting agent, or a pharmaceutically acceptable salt thereof; and an agent capable of sensitizing or potentiating induction of the lytic phase of the virus, or a pharmaceutically acceptable salt thereof, for use in the induction of a lytic phase of a virus. In another aspect, the present disclosure provides a dual HDAC3 and HDAC8 targeting agent, or a pharmaceutically acceptable salt thereof; and an agent capable of sensitizing or potentiating induction of the lytic phase of the virus, or a pharmaceutically acceptable salt thereof, for use in the manufacture of a medicament for the induction of a lytic phase of a virus.

In another aspect, the present disclosure provides a dual HDAC3 and HDAC8 targeting agent, or a pharmaceutically acceptable salt thereof; and an agent capable of sensitizing or potentiating induction of the lytic phase of the virus, or a pharmaceutically acceptable salt thereof, for use in the reactivation of a virus. In another aspect, the present disclosure provides a dual HDAC3 and HDAC8 targeting agent, or a pharmaceutically acceptable salt thereof; and an agent capable of sensitizing or potentiating induction of the lytic phase of the virus, or a pharmaceutically acceptable salt thereof, for use in the manufacture of a medicament for the reactivation of a virus.

### Additional Embodiments of Methods of Inhibiting Cell Proliferation or Promoting Apoptosis, Inducing a Lytic Phase of a Virus, and Reactivating a Virus

In some embodiments, the dual HDAC3 and HDAC8 targeting agent is YX968, or a pharmaceutically acceptable salt thereof. In some embodiments, the dual HDAC3 and HDAC8 targeting agent is a pharmaceutically acceptable salt of YX968. In some embodiments, the dual HDAC3 and HDAC8 targeting agent is YX968.

In some embodiments, the dual HDAC3 and HDAC8 targeting agent is:

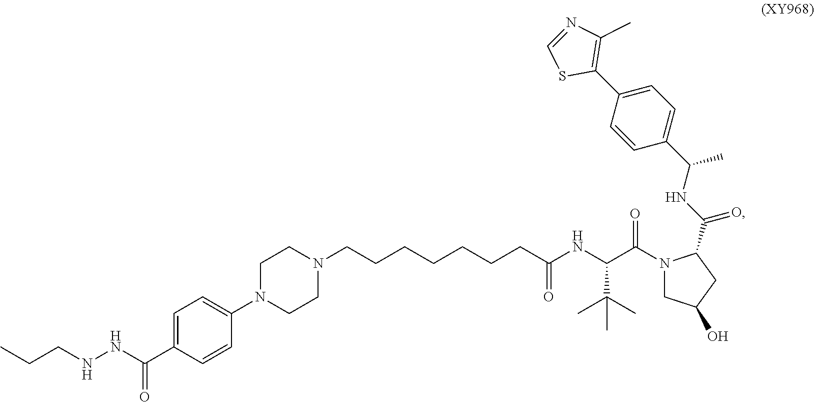 [SMILES: CCCNNC(=O)c1ccc(N2CCN(CCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

or a pharmaceutically acceptable salt thereof. In some embodiments, the dual HDAC3 and HDAC8 targeting agent is a pharmaceutically acceptable salt of:

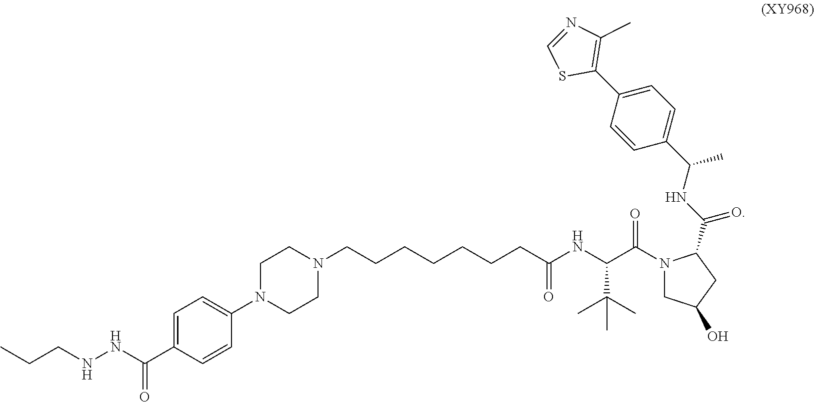 [SMILES: CCCNNC(=O)c1ccc(N2CCN(CCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

In some embodiments, the dual HDAC3 and HDAC8 targeting agent is:

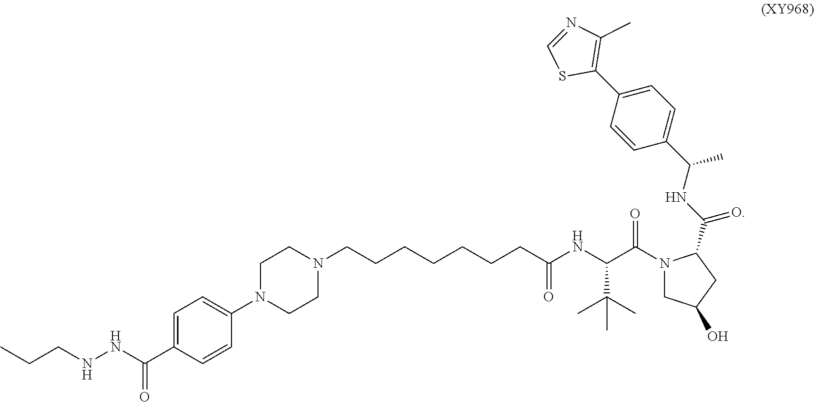 [SMILES: CCCNNC(=O)c1ccc(N2CCN(CCCCCCCC(=O)N[C@H](C(=O)N3C[C@H](O)C[C@H]3C(=O)N[C@@H](C)c3ccc(-c4scnc4C)cc3)C(C)(C)C)CC2)cc1]

YX968 and other molecules that induce the lytic phase can potentially be combined into a pharmaceutical composition that contains molecules that inhibit EBV from creating infectious virions, such as ganciclovir, cidofovir, acyclovir, and foscarnet. New formulations of these compositions, or compositions that similarly incorporate into the cell and viral genome to block genome replication (i.e., are used for the same purpose) are within the scope of the present disclosure.

In some embodiments, the antiviral agent is ganciclovir, cidofovir, acyclovir, or foscarnet, or a pharmaceutically acceptable salt thereof. In some embodiments, the antiviral agent is a pharmaceutically acceptable salt of ganciclovir, cidofovir, acyclovir, or foscarnet. In some embodiments, the antiviral agent is ganciclovir, cidofovir, acyclovir, or foscarnet.

In some embodiments, the antiviral agent is ganciclovir, or a pharmaceutically acceptable salt thereof. In some embodiments, the antiviral agent is a pharmaceutically acceptable salt of ganciclovir. In some embodiments, the antiviral agent is ganciclovir.

In some embodiments, the antiviral agent is cidofovir, or a pharmaceutically acceptable salt thereof. In some embodiments, the antiviral agent is a pharmaceutically acceptable salt of cidofovir. In some embodiments, the antiviral agent is cidofovir.

In some embodiments, the antiviral agent is acyclovir, or a pharmaceutically acceptable salt thereof. In some embodiments, the antiviral agent is a pharmaceutically acceptable salt of acyclovir. In some embodiments, the antiviral agent is acyclovir.

In some embodiments, the antiviral agent is foscarnet, or a pharmaceutically acceptable salt thereof. In some embodiments, the antiviral agent is a pharmaceutically acceptable salt of foscarnet. In some embodiments, the antiviral agent is foscarnet.

In some embodiments, the dual HDAC3 and HDAC8 targeting agent is YX968, or a pharmaceutically acceptable salt thereof; and the antiviral agent is ganciclovir, cidofovir, acyclovir, or foscarnet, or a pharmaceutically acceptable salt thereof. In some embodiments, the dual HDAC3 and HDAC8 targeting agent is YX968, or a pharmaceutically acceptable salt thereof; and the antiviral agent is a pharmaceutically acceptable salt of ganciclovir, cidofovir, acyclovir, or foscarnet. In some embodiments, the dual HDAC3 and HDAC8 targeting agent is YX968, or a pharmaceutically acceptable salt thereof; and the antiviral agent is ganciclovir, cidofovir, acyclovir, or foscarnet.

In some embodiments, the dual HDAC3 and HDAC8 targeting agent is a pharmaceutically acceptable salt of YX968; and the antiviral agent is ganciclovir, cidofovir, acyclovir, or foscarnet, or a pharmaceutically acceptable salt thereof. In some embodiments, the dual HDAC3 and HDAC8 targeting agent is a pharmaceutically acceptable salt of YX968; and the antiviral agent is a pharmaceutically acceptable salt of ganciclovir, cidofovir, acyclovir, or foscarnet. In some embodiments, the dual HDAC3 and HDAC8 targeting agent is a pharmaceutically acceptable salt of YX968; and the antiviral agent is ganciclovir, cidofovir, acyclovir, or foscarnet.

In some embodiments, the dual HDAC3 and HDAC8 targeting agent is YX968; and the antiviral agent is ganciclovir, cidofovir, acyclovir, or foscarnet, or a pharmaceutically acceptable salt thereof. In some embodiments, the dual HDAC3 and HDAC8 targeting agent is YX968; and the antiviral agent is a pharmaceutically acceptable salt of ganciclovir, cidofovir, acyclovir, or foscarnet. In some embodiments, the dual HDAC3 and HDAC8 targeting agent is YX968; and the antiviral agent is ganciclovir, cidofovir, acyclovir, or foscarnet.

In some embodiments, the dual HDAC3 and HDAC8 targeting agent is YX968, or a pharmaceutically acceptable salt thereof; and the antiviral agent is ganciclovir, or a pharmaceutically acceptable salt thereof. In some embodiments, the dual HDAC3 and HDAC8 targeting agent is YX968, or a pharmaceutically acceptable salt thereof; and the antiviral agent is a pharmaceutically acceptable salt of ganciclovir. In some embodiments, the dual HDAC3 and HDAC8 targeting agent is YX968, or a pharmaceutically acceptable salt thereof; and the antiviral agent is ganciclovir.

In some embodiments, the dual HDAC3 and HDAC8 targeting agent is a pharmaceutically acceptable salt of YX968; and the antiviral agent is ganciclovir, or a pharmaceutically acceptable salt thereof. In some embodiments, the dual HDAC3 and HDAC8 targeting agent is a pharmaceutically acceptable salt of YX968; and the antiviral agent is a pharmaceutically acceptable salt of ganciclovir. In some embodiments, the dual HDAC3 and HDAC8 targeting agent is a pharmaceutically acceptable salt of YX968; and the antiviral agent is ganciclovir.

In some embodiments, the dual HDAC3 and HDAC8 targeting agent is YX968; and the antiviral agent is ganciclovir, or a pharmaceutically acceptable salt thereof. In some embodiments, the dual HDAC3 and HDAC8 targeting agent is YX968; and the antiviral agent is a pharmaceutically acceptable salt of ganciclovir. In some embodiments, the dual HDAC3 and HDAC8 targeting agent is YX968; and the antiviral agent is ganciclovir.

In some embodiments, the dual HDAC3 and HDAC8 targeting agent is YX968, or a pharmaceutically acceptable salt thereof; and the antiviral agent is cidofovir, or a pharmaceutically acceptable salt thereof. In some embodiments, the dual HDAC3 and HDAC8 targeting agent is YX968, or a pharmaceutically acceptable salt thereof; and the antiviral agent is a pharmaceutically acceptable salt of cidofovir. In some embodiments, the dual HDAC3 and HDAC8 targeting agent is YX968, or a pharmaceutically acceptable salt thereof; and the antiviral agent is cidofovir.

In some embodiments, the dual HDAC3 and HDAC8 targeting agent is a pharmaceutically acceptable salt of YX968; and the antiviral agent is cidofovir, or a pharmaceutically acceptable salt thereof. In some embodiments, the dual HDAC3 and HDAC8 targeting agent is a pharmaceutically acceptable salt of YX968; and the antiviral agent is a pharmaceutically acceptable salt of cidofovir. In some embodiments, the dual HDAC3 and HDAC8 targeting agent is a pharmaceutically acceptable salt of YX968; and the antiviral agent is cidofovir.

In some embodiments, the dual HDAC3 and HDAC8 targeting agent is YX968; and the antiviral agent is cidofovir, or a pharmaceutically acceptable salt thereof. In some embodiments, the dual HDAC3 and HDAC8 targeting agent is YX968; and the antiviral agent is a pharmaceutically acceptable salt of cidofovir. In some embodiments, the dual HDAC3 and HDAC8 targeting agent is YX968; and the antiviral agent is cidofovir.

In some embodiments, the dual HDAC3 and HDAC8 targeting agent is YX968, or a pharmaceutically acceptable salt thereof; and the antiviral agent is acyclovir, or a pharmaceutically acceptable salt thereof. In some embodiments, the dual HDAC3 and HDAC8 targeting agent is YX968, or a pharmaceutically acceptable salt thereof; and the antiviral agent is a pharmaceutically acceptable salt of acyclovir. In some embodiments, the dual HDAC3 and HDAC8 targeting agent is YX968, or a pharmaceutically acceptable salt thereof; and the antiviral agent is acyclovir.

In some embodiments, the dual HDAC3 and HDAC8 targeting agent is a pharmaceutically acceptable salt of YX968; and the antiviral agent is acyclovir, or a pharmaceutically acceptable salt thereof. In some embodiments, the dual HDAC3 and HDAC8 targeting agent is a pharmaceutically acceptable salt of YX968; and the antiviral agent is a pharmaceutically acceptable salt of acyclovir. In some embodiments, the dual HDAC3 and HDAC8 targeting agent is a pharmaceutically acceptable salt of YX968; and the antiviral agent is acyclovir.

In some embodiments, the dual HDAC3 and HDAC8 targeting agent is YX968; and the antiviral agent is acyclovir, or a pharmaceutically acceptable salt thereof. In some embodiments, the dual HDAC3 and HDAC8 targeting agent is YX968; and the antiviral agent is a pharmaceutically acceptable salt of acyclovir. In some embodiments, the dual HDAC3 and HDAC8 targeting agent is YX968; and the antiviral agent is acyclovir.

In some embodiments, the dual HDAC3 and HDAC8 targeting agent is YX968, or a pharmaceutically acceptable salt thereof; and the antiviral agent is foscarnet, or a pharmaceutically acceptable salt thereof. In some embodiments, the dual HDAC3 and HDAC8 targeting agent is YX968, or a pharmaceutically acceptable salt thereof; and the antiviral agent is a pharmaceutically acceptable salt of foscarnet. In some embodiments, the dual HDAC3 and HDAC8 targeting agent is YX968, or a pharmaceutically acceptable salt thereof; and the antiviral agent is foscarnet.

In some embodiments, the dual HDAC3 and HDAC8 targeting agent is a pharmaceutically acceptable salt of YX968; and the antiviral agent is foscarnet, or a pharmaceutically acceptable salt thereof. In some embodiments, the dual HDAC3 and HDAC8 targeting agent is a pharmaceutically acceptable salt of YX968; and the antiviral agent is a pharmaceutically acceptable salt of foscarnet. In some embodiments, the dual HDAC3 and HDAC8 targeting agent is a pharmaceutically acceptable salt of YX968; and the antiviral agent is foscarnet.

In some embodiments, the dual HDAC3 and HDAC8 targeting agent is YX968; and the antiviral agent is foscarnet, or a pharmaceutically acceptable salt thereof. In some embodiments, the dual HDAC3 and HDAC8 targeting agent is YX968; and the antiviral agent is a pharmaceutically acceptable salt of foscarnet. In some embodiments, the dual HDAC3 and HDAC8 targeting agent is YX968; and the antiviral agent is foscarnet.

In some embodiments, the additional agent capable of sensitizing or potentiating induction of the lytic phase of the virus is an EZH2 inhibitor (e.g., tazemetostat). In some embodiments, the additional agent capable of sensitizing or potentiating induction of the lytic phase of the virus is tazemetostat, or a pharmaceutically acceptable salt thereof. In some embodiments, the additional agent capable of sensitizing or potentiating induction of the lytic phase of the virus is tazemetostat. In some embodiments, the additional agent capable of sensitizing or potentiating induction of the lytic phase of the virus is a pharmaceutically acceptable salt of tazemetostat.

In some embodiments, the dual HDAC3 and HDAC8 targeting agent is YX968, or a pharmaceutically acceptable salt thereof; and the additional agent capable of sensitizing or potentiating induction of the lytic phase of the virus is tazemetostat, or a pharmaceutically acceptable salt thereof. In some embodiments, the dual HDAC3 and HDAC8 targeting agent is YX968, or a pharmaceutically acceptable salt thereof; and the additional agent capable of sensitizing or potentiating induction of the lytic phase of the virus is a pharmaceutically acceptable salt of tazemetostat. In some embodiments, the dual HDAC3 and HDAC8 targeting agent is YX968, or a pharmaceutically acceptable salt thereof; and the additional agent capable of sensitizing or potentiating induction of the lytic phase of the virus is tazemetostat.

In some embodiments, the dual HDAC3 and HDAC8 targeting agent is a pharmaceutically acceptable salt of YX968; and the additional agent capable of sensitizing or potentiating induction of the lytic phase of the virus is tazemetostat, or a pharmaceutically acceptable salt thereof. In some embodiments, the dual HDAC3 and HDAC8 targeting agent is a pharmaceutically acceptable salt of YX968; and the additional agent capable of sensitizing or potentiating induction of the lytic phase of the virus is a pharmaceutically acceptable salt of tazemetostat. In some embodiments, the dual HDAC3 and HDAC8 targeting agent is a pharmaceutically acceptable salt of YX968; and the additional agent capable of sensitizing or potentiating induction of the lytic phase of the virus is tazemetostat.

In some embodiments, the dual HDAC3 and HDAC8 targeting agent is YX968; and the additional agent capable of sensitizing or potentiating induction of the lytic phase of the virus is tazemetostat, or a pharmaceutically acceptable salt thereof. In some embodiments, the dual HDAC3 and HDAC8 targeting agent is YX968; and the additional agent capable of sensitizing or potentiating induction of the lytic phase of the virus is a pharmaceutically acceptable salt of tazemetostat. In some embodiments, the dual HDAC3 and HDAC8 targeting agent is YX968; and the additional agent capable of sensitizing or potentiating induction of the lytic phase of the virus is tazemetostat.

In some embodiments, the method further comprises administering to the subject in need thereof an effective amount of an additional agent capable of inducing the lytic phase of the virus, or a pharmaceutically acceptable salt thereof. In some embodiments, the pharmaceutical composition further comprises an effective amount of an additional agent capable of inducing the lytic phase of the virus, or a pharmaceutically acceptable salt thereof.

In some embodiments, the additional agent capable of inducing the lytic phase of the virus is an HDAC inhibitor, a DNA methyltransferase inhibitor, a phorbol ester, a hypoxia-mimicking agent, or an immunoglobulin. In some embodiments, the additional agent capable of inducing the lytic phase of the virus is an HDAC inhibitor. In some embodiments, the additional agent capable of inducing the lytic phase of the virus is a DNA methyltransferase inhibitor. In some embodiments, the additional agent capable of inducing the lytic phase of the virus is a phorbol ester. In some embodiments, the additional agent capable of inducing the lytic phase of the virus is a hypoxia-mimicking agent. In some embodiments, the additional agent capable of inducing the lytic phase of the virus is an immunoglobulin.

In some embodiments, the additional agent capable of inducing the lytic phase of the virus is sodium butyrate, valproic acid, romidepsin, MS-275, apicidin, oxamflatin, Scriptaid, panobinostat, or nanatinostat, or a pharmaceutically acceptable salt thereof. In some embodiments, the additional agent capable of inducing the lytic phase of the virus is sodium butyrate, or a pharmaceutically acceptable salt thereof. In some embodiments, the additional agent capable of inducing the lytic phase of the virus is valproic acid, or a pharmaceutically acceptable salt thereof. In some embodiments, the additional agent capable of inducing the lytic phase of the virus is romidepsin, or a pharmaceutically acceptable salt thereof. In some embodiments, the additional agent capable of inducing the lytic phase of the virus is MS-275, or a pharmaceutically acceptable salt thereof. In some embodiments, the additional agent capable of inducing the lytic phase of the virus is apicidin, or a pharmaceutically acceptable salt thereof. In some embodiments, the additional agent capable of inducing the lytic phase of the virus is oxamflatin, or a pharmaceutically acceptable salt thereof. In some embodiments, the additional agent capable of inducing the lytic phase of the virus is Scriptaid, or a pharmaceutically acceptable salt thereof. In some embodiments, the additional agent capable of inducing the lytic phase of the virus is Panobinostat, or a pharmaceutically acceptable salt thereof. In some embodiments, the additional agent capable of inducing the lytic phase of the virus is nanatinostat, or a pharmaceutically acceptable salt thereof.

In some embodiments, the additional agent capable of inducing the lytic phase of the virus is sodium butyrate, valproic acid, romidepsin, MS-275, apicidin, oxamflatin, Scriptaid, panobinostat, or nanatinostat. In some embodiments, the additional agent capable of inducing the lytic phase of the virus is sodium butyrate. In some embodiments, the additional agent capable of inducing the lytic phase of the virus is valproic acid. In some embodiments, the additional agent capable of inducing the lytic phase of the virus is romidepsin. In some embodiments, the additional agent capable of inducing the lytic phase of the virus is MS-275. In some embodiments, the additional agent capable of inducing the lytic phase of the virus is apicidin. In some embodiments, the additional agent capable of inducing the lytic phase of the virus is oxamflatin. In some embodiments, the additional agent capable of inducing the lytic phase of the virus is Scriptaid. In some embodiments, the additional agent capable of inducing the lytic phase of the virus is panobinostat. In some embodiments, the additional agent capable of inducing the lytic phase of the virus is nanatinostat.

In some embodiments, the virus is a herpesvirus. In some embodiments, the virus is a gamma-herpesvirus. In some embodiments, the virus is Epstein-Barr virus (EBV) or Kaposi's sarcoma-associated herpesvirus (KSHV). In some embodiments, the virus is Epstein-Barr virus (EBV). In some embodiments, the virus is Kaposi's sarcoma-associated herpesvirus (KSHV).

In some embodiments, the virus is in the latent state or the abortive lytic state. In some embodiments, the virus is in the latent state. In some embodiments, the virus is in the abortive lytic state.

In some embodiments, the subject in need thereof has not been administered one or more additional pharmaceutical agents and/or radiation. In some embodiments, the subject in need thereof has not been administered one or more additional pharmaceutical agents and radiation. In some embodiments, the subject in need thereof has not been administered one or more additional pharmaceutical agents or radiation. In some embodiments, the subject in need thereof has not been administered one or more additional pharmaceutical agents. In some embodiments, the subject in need thereof has not been administered radiation.

In some embodiments, the subject in need thereof has been administered one or more additional pharmaceutical agents and/or radiation. In some embodiments, the subject in need thereof has been administered one or more additional pharmaceutical agents and radiation. In some embodiments, the subject in need thereof has been administered one or more additional pharmaceutical agents or radiation. In some embodiments, the subject in need thereof has been administered one or more additional pharmaceutical agents. In some embodiments, the subject in need thereof has been administered radiation.

In some embodiments, the method further comprises administering to the subject in need thereof one or more additional pharmaceutical agents and/or radiation. In some embodiments, the method further comprises administering to the subject in need thereof one or more additional pharmaceutical agents and radiation. In some embodiments, the method further comprises administering to the subject in need thereof one or more additional pharmaceutical agents or radiation. In some embodiments, the method further comprises administering to the subject in need thereof one or more additional pharmaceutical agents. In some embodiments, the method further comprises administering to the subject in need thereof radiation.

In some embodiments, the one or more additional pharmaceutical agents are chemotherapeutic agents and/or PARP inhibitors. In some embodiments, the one or more additional pharmaceutical agents are chemotherapeutic agents and PARP inhibitors. In some embodiments, the one or more additional pharmaceutical agents are chemotherapeutic agents or PARP inhibitors. In some embodiments, the one or more additional pharmaceutical agents are chemotherapeutic agents. In some embodiments, the one or more additional pharmaceutical agents are PARP inhibitors. In some embodiments, the one or more additional pharmaceutical agents are CODOX-M/IVAC.

An EBV-positive LPD or EBV-associated malignancy or pre-malignancy in an immunosuppressed or non-immunosuppressed subject can be treated according to methods described herein. Cancer cells that can be killed or their proliferation inhibited include, but are not limited to, iatrogenic lymphoproliferative disorder/lymphomas, post-transplant lymphoproliferative disorder (PTLD)/lymphomas, Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cell carcinoma, AIDS-related ymphomas, chronic active EBV infection (CAEBV), and EBV-positive gastric carcinomas.

In some embodiments, the subject in need thereof is non-immunosuppressed. In some embodiments, the subject in need thereof has not been administered an immunosuppressive or immunomodulatory agent.

In some embodiments, the subject in need thereof is immunosuppressed. In some embodiments, the subject in need thereof has been administered an immunosuppressive or immunomodulatory agent.

In some embodiments, the method further comprises administering to the subject in need thereof an immunosuppressive or immunomodulatory agent. In some embodiments, the immunosuppressive or immunomodulatory agent is selected from corticosteroids (e.g., prednisone (Deltasone, Orasone), budesonide (Entocort EC), prednisolone (Millipred)), Janus kinase inhibitors (e.g., tofacitinib (Xeljanz)), calcineurin inhibitors (e.g., cyclosporine (Neoral, Sandimmune, SangCya), tacrolimus (Astagraf XL, Envarsus XR, Prograf)), mTOR inhibitors (e.g., sirolimus (Rapamune), everolimus (Afinitor, Zortress), inosine monophosphate dehydrogenase (IMDH) inhibitors (e.g., azathioprine (Azasan, Imuran), leflunomide (Arava), mycophenolate (CellCept, Myfortic), biologics (e.g., antibody, monoclonal antibody) (e.g., abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta)), and antimetabolites (e.g., methotrexate).

In some embodiments, the immunosuppressive or immunomodulatory agent is a corticosteroid (e.g., prednisone (Deltasone, Orasone), budesonide (Entocort EC), prednisolone (Millipred)). In some embodiments, the corticosteroid is prednisone (Deltasone, Orasone). In some embodiments, the corticosteroid is budesonide (Entocort EC). In some embodiments, the corticosteroid is prednisolone (Millipred). In some embodiments, the immunosuppressive or immunomodulatory agent is a Janus kinase inhibitor (e.g., tofacitinib (Xeljanz)). In some embodiments, the Janus kinase inhibitor is tofacitinib (Xeljanz). In some embodiments, the immunosuppressive or immunomodulatory agent is a calcineurin inhibitor (e.g., cyclosporine (Neoral, Sandimmune, SangCya). In some embodiments, the calcineurin inhibitor is cyclosporine (Neoral, Sandimmune, SangCya). In some embodiments, the immunosuppressive or immunomodulatory agent is tacrolimus (Astagraf XL, Envarsus XR, Prograf)). In some embodiments, the immunosuppressive or immunomodulatory agent is an mTOR inhibitor (e.g., sirolimus (Rapamune), everolimus (Afinitor, Zortress). In some embodiments, the mTOR inhibitor is sirolimus (Rapamune). In some embodiments, the mTOR inhibitor is everolimus (Afinitor, Zortress). In some embodiments, the immunosuppressive or immunomodulatory agent is an inosine monophosphate dehydrogenase (IMDH) inhibitor (e.g., azathioprine (Azasan, Imuran), leflunomide (Arava), mycophenolate (CellCept, Myfortic). In some embodiments, the IMDH inhibitor is azathioprine (Azasan, Imuran). In some embodiments, the IMDH inhibitor is leflunomide (Arava). In some embodiments, the IMDH inhibitor is mycophenolate (CellCept, Myfortic). In some embodiments, the immunosuppressive or immunomodulatory agent is a biologic (e.g., abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta)). In some embodiments, the biologic is abatacept (Orencia). In some embodiments, the biologic is adalimumab (Humira). In some embodiments, the biologic is anakinra (Kineret). In some embodiments, the biologic is certolizumab (Cimzia). In some embodiments, the biologic is etanercept (Enbrel). In some embodiments, the biologic is golimumab (Simponi). In some embodiments, the biologic is infliximab (Remicade). In some embodiments, the biologic is ixekizumab (Taltz). In some embodiments, the biologic is natalizumab (Tysabri). In some embodiments, the biologic is rituximab (Rituxan). In some embodiments, the biologic is secukinumab (Cosentyx). In some embodiments, the biologic is tocilizumab (Actemra). In some embodiments, the biologic is ustekinumab (Stelara). In some embodiments, the biologic is vedolizumab (Entyvio). In some embodiments, the biologic is basiliximab (Simulect). In some embodiments, the biologic is daclizumab (Zinbryta). In some embodiments, the immunosuppressive or immunomodulatory agent is an antimetabolite (e.g., methotrexate). In some embodiments, the antimetabolite is methotrexate.

In some embodiments, the method prevents or reduces the risk of developing EBV+ LPD in a subject that has been on or is currently on T cell suppressive therapy or anti-TNF alpha therapy. The T cell suppressive therapy can be, but is not limited to, antithymocyte globulin or recombinant antithymocyte globulin therapy.

In some embodiments, the subject has been administered one or more of T cell suppressive therapy and anti-TNF alpha therapy.

In some embodiments, the T cell suppressive therapy is antithymocyte globulin or recombinant antithymocyte globulin therapy. In some embodiments, the T cell suppressive therapy is antithymocyte globulin therapy. In some embodiments, the T cell suppressive therapy is recombinant antithymocyte globulin therapy.

In some embodiments, the subject in need thereof has a lymphoproliferative disease. In some embodiments, the subject in need thereof has a lymphoproliferative disease (LPD). In some embodiments, the subject in need thereof has an EBV-positive lymphoproliferative disease (LPD). In some embodiments, the subject in need thereof has an KSHV-positive lymphoproliferative disease (LPD).

In some embodiments, the method prevents the subject in need thereof from developing a lymphoproliferative disease (LPD). In some embodiments, the method prevents the subject in need thereof from developing an EBV-positive lymphoproliferative disease (LPD). In some embodiments, the method prevents the subject in need thereof from developing an KSHV-positive lymphoproliferative disease (LPD).

In some embodiments, the method prevents the subject in need thereof from developing an EBV-positive lymphoproliferative disease (LPD), malignancy, or pre-malignancy, wherein the subject in need thereof is a transplant recipient. Lymphoproliferative diseases that can be prevented or for which the risk can be reduced include, but are not limited to, iatrogenic lymphoproliferative disorder/lymphomas, post- transplant lymphoproliferative disorder (PTLD)/lymphomas, Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cell carcinoma, AIDS-related lymphomas, chronic active EBV infection (CAEBV), and EBV-positive gastric carcinomas.

In some embodiments, the subject in need thereof is a transplant recipient. In some embodiments, the transplant is a solid organ transplant or a hematopoietic transplant. In some embodiments, the transplant is a solid organ transplant. In some embodiments, the transplant is a hematopoietic transplant.

In some embodiments, the methods can be used to treat persons who have lymphoproliferative diseases despite evidence of elimination of the malignant cells. In some embodiments the therapeutic value will be in halting the continued expansion of proliferative cells rather than eliminating them completely.

In some embodiments, the method comprises continuing administration until the subject in need thereof is in remission from a cancer. In some embodiments, the cancer is iatrogenic lymphoma, post-transplant lymphoma, Burkitt lymphoma, B-cell non-Hodgkin lymphoma, diffuse large B-cell lymphoma (DLBCL), central nervous system (CNS) lymphoma, NK/T-cell lymphoma, T cell non-Hodgkin lymphoma, or Hodgkin's lymphoma. In some embodiments, the cancer is EBV-positive gastric carcinoma or nasopharyngeal cell carcinoma. In some embodiments, the cancer is oral hairy leukoplakia, central nervous system (CNS) lymphoma, or AIDS-related lymphoma.

In some embodiments, the subject in need thereof comprises EBV-positive cells. In some embodiments, the subject in need thereof comprises one or more of EBV-positive cancer cells and EBV-positive pre-cancerous cells. In some embodiments, the subject in need thereof comprises EBV-positive cancer cells and EBV-positive pre-cancerous cells. In some embodiments, the subject in need thereof comprises EBV-positive cancer cells. In some embodiments, the subject in need thereof comprises EBV-positive pre-cancerous cells.

In some embodiments, the EBV is in a latent lytic state or abortive lytic state. In some embodiments, the EBV is in a latent lytic state. In some embodiments, the EBV is in an abortive lytic state. In some embodiments, the method comprises inducing a conversion of EBV from an abortive lytic state to a lytic state. In some embodiments, the method comprises inducing a conversion of EBV from a latent state to a lytic state.

In some embodiments, the method comprises administering the dual HDAC3 and HDAC8 targeting agent, or the pharmaceutical composition comprising the dual HDAC3 and HDAC8 targeting agent, at the same time as the presence of EBV+ cells with EBV in the latent or abortive lytic state is established. In some embodiments, the method comprises administering the dual HDAC3 and HDAC8 targeting agent, or the pharmaceutical composition comprising the dual HDAC3 and HDAC8 targeting agent, after the presence of EBV+ cells with EBV in the latent or abortive lytic state has been established. In some embodiments, the method comprises administering the dual HDAC3 and HDAC8 targeting agent, or the pharmaceutical composition comprising the dual HDAC3 and HDAC8 targeting agent, until the EBV+ cells with EBV in the latent or abortive lytic state have been eliminated.

In some embodiments, EBV levels in the subject in need thereof are monitored, and the pharmaceutical composition containing a dual HDAC3 and HDAC8 targeting agent (such as YX968) is administered to the subject in need thereof if the levels of EBV in the subject increase.

In some embodiments, the method further comprises determining an initial level of EBV in the subject in need thereof; and determining at a later time a subsequent level of EBV in the subject in need thereof; wherein the administration occurs when the subsequent level of EBV is greater than the initial level of EBV.

In some embodiments, the subject in need thereof comprises KSHV-positive cells. In some embodiments, the subject in need thereof comprises one or more of KSHV-positive cancer cells and KSHV-positive pre-cancerous cells. In some embodiments, the subject in need thereof comprises KSHV-positive cancer cells and KSHV-positive pre-cancerous cells. In some embodiments, the subject in need thereof comprises KSHV-positive cancer cells. In some embodiments, the subject in need thereof comprises KSHV-positive pre-cancerous cells.

In some embodiments, the KSHV is in a latent lytic state or abortive lytic state. In some embodiments, the KSHV is in a latent lytic state. In some embodiments, the KSHV is in an abortive lytic state. In some embodiments, the method comprises inducing a conversion of KSHV from an abortive lytic state to a lytic state. In some embodiments, the method comprises inducing a conversion of KSHV from a latent state to a lytic state.

In some embodiments, the method comprises administering the dual HDAC3 and HDAC8 targeting agent, or the pharmaceutical composition comprising the dual HDAC3 and HDAC8 targeting agent, at the same time as the presence of KSHV+ cells with KSHV in the latent or abortive lytic state is established. In some embodiments, the method comprises administering the dual HDAC3 and HDAC8 targeting agent, or the pharmaceutical composition comprising the dual HDAC3 and HDAC8 targeting agent, after the presence of KSHV+ cells with KSHV in the latent or abortive lytic state has been established. In some embodiments, the method comprises administering the dual HDAC3 and HDAC8 targeting agent, or the pharmaceutical composition comprising the dual HDAC3 and HDAC8 targeting agent, until the KSHV+ cells with KSHV in the latent or abortive lytic state have been eliminated.

In some embodiments, KSHV levels in the subject in need thereof are monitored, and the pharmaceutical composition containing a dual HDAC3 and HDAC8 targeting agent (such as YX968) is administered to the subject in need thereof if the levels of KSHV in the subject increase.

In some embodiments, the method further comprises determining an initial level of KSHV in the subject in need thereof; and determining at a later time a subsequent level of KSHV in the subject in need thereof; wherein the administration occurs when the subsequent level of KSHV is greater than the initial level of KSHV.

In some embodiments, the subject in need thereof has been identified as being in need of treatment of the disease associated with the virus.

In some embodiments, the subject suffers from a disease.

In some embodiments, the disease is a proliferative disease. In some embodiments, the proliferative disease is a lymphoproliferative disease. In some embodiments, the lymphoproliferative disease is iatrogenic lymphoproliferative disorder, iatrogenic lymphoma, post-transplant lymphoproliferative disorder (PTLD), post-transplant lymphoma, Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cell carcinoma, AIDS-related lymphoma, chronic active EBV infection (CAEBV), hemophagocytic lymphohistiocytosis, or EBV-positive gastric carcinoma.

In some embodiments, the lymphoproliferative disease is iatrogenic lymphoproliferative disorder. In some embodiments, the lymphoproliferative disease is iatrogenic lymphoma. In some embodiments, the lymphoproliferative disease is post-transplant lymphoproliferative disorder (PTLD). In some embodiments, the lymphoproliferative disease is post-transplant lymphoma. In some embodiments, the lymphoproliferative disease is Burkitt lymphoma. In some embodiments, the lymphoproliferative disease is B-cell non-Hodgkin lymphoma. In some embodiments, the lymphoproliferative disease is diffuse large B-cell lymphoma (DLBCL). In some embodiments, the lymphoproliferative disease is CNS lymphoma. In some embodiments, the lymphoproliferative disease is NK/T-cell lymphoma. In some embodiments, the lymphoproliferative disease is T cell non-Hodgkin lymphoma. In some embodiments, the lymphoproliferative disease is Hodgkin lymphoma. In some embodiments, the lymphoproliferative disease is nasopharyngeal cell carcinoma. In some embodiments, the lymphoproliferative disease is AIDS-related lymphoma. In some embodiments, the lymphoproliferative disease is chronic active EBV infection (CAEBV). In some embodiments, the lymphoproliferative disease is hemophagocytic lymphohistiocytosis. In some embodiments, the lymphoproliferative disease is EBV-positive gastric carcinoma.

In some embodiments, the proliferative disease is a cancer. In some embodiments, the cancer is a lymphoid malignancy. In some embodiments, the cancer is iatrogenic lymphoma, post-transplant lymphoma, Burkitt lymphoma, B-cell non-Hodgkin lymphoma, diffuse large B-cell lymphoma (DLBCL), central nervous system (CNS) lymphoma, NK/T-cell lymphoma, T cell non-Hodgkin lymphoma, or Hodgkin's lymphoma.

In some embodiments, the cancer is iatrogenic lymphoma. In some embodiments, the cancer is post-transplant lymphoma. In some embodiments, the cancer is Burkitt lymphoma. In some embodiments, the cancer is B-cell non-Hodgkin lymphoma. In some embodiments, the cancer is diffuse large B-cell lymphoma (DLBCL). In some embodiments, the cancer is central nervous system (CNS) lymphoma. In some embodiments, the cancer is NK/T-cell lymphoma. In some embodiments, the cancer is T cell non-Hodgkin lymphoma. In some embodiments, the cancer is Hodgkin's lymphoma.

In some embodiments, the cancer is a non-lymphoid malignancy. In some embodiments, the cancer is EBV-positive gastric carcinoma or nasopharyngeal cell carcinoma. In some embodiments, the cancer is EBV-positive gastric carcinoma. In some embodiments, the cancer is EBV-positive nasopharyngeal cell carcinoma.

In some embodiments, the cancer is associated with human immunodeficiency virus. In some embodiments, the cancer is oral hairy leukoplakia, central nervous system (CNS) lymphoma, or AIDS-related lymphoma. In some embodiments, the cancer is oral hairy leukoplakia. In some embodiments, the cancer is central nervous system (CNS) lymphoma. In some embodiments, the cancer is AIDS-related lymphoma.

In some embodiments, the disease is chronic active EBV infection (CAEBV).

Persons with multiple sclerosis (MS) may also benefit from the killing of their EBV positive B cells, as MS has been causally linked to EBV.

In some embodiments, the disease is multiple sclerosis (MS).

In some embodiments, the disease is associated with Kaposi's sarcoma-associated herpesvirus (KSHV).

In some embodiments, the disease is a primary effusion lymphoma (PEL), multicentric Castleman's disease, or Kaposi's sarcoma. In some embodiments, the disease is a primary effusion lymphoma (PEL). In some embodiments, the disease is multicentric Castleman's disease. In some embodiments, the disease is Kaposi's sarcoma.

In some embodiments, the dual HDAC3 and HDAC8 targeting agent, or pharmaceutically acceptable salt thereof, degrades HDAC3. In some embodiments, the dual HDAC3 and HDAC8 targeting agent, or pharmaceutically acceptable salt thereof, degrades HDAC8. In some embodiments, the dual HDAC3 and HDAC8 targeting agent, or pharmaceutically acceptable salt thereof, inhibits HDAC2.

In some embodiments, the method comprises increasing an activating histone mark at the BZLF1 promoter. In some embodiments, the activating histone mark is H3K27Ac.

### Pharmaceutical Compositions and Kits

The present disclosure provides pharmaceutical compositions comprising a compound disclosed herein, or a pharmaceutically acceptable salt thereof, and optionally a pharmaceutically acceptable excipient. In certain embodiments, the pharmaceutical composition described herein comprises a compound of disclosed herein, or a pharmaceutically acceptable salt thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a disease associated with Epstein-Barr virus (EBV) in a subject in need thereof. In certain embodiments, the effective amount is an amount effective for treating a disease associated with Kaposi's sarcoma-associated herpesvirus (KSHV) in a subject in need thereof. In certain embodiments, the effective amount is an amount effective for treating a proliferative disease (e.g., a lymphoproliferative disease, cancer (e.g., a lymphoid malignancy, a non-lymphoid malignancy, cancer associated with human immunodeficiency virus)) in a subject in need thereof. In certain embodiments, the effective amount is an amount effective for preventing a proliferative disease (e.g., a lymphoproliferative disease, cancer (e.g., a lymphoid malignancy, a non-lymphoid malignancy, cancer associated with human immunodeficiency virus)) in a subject in need thereof. In certain embodiments, the effective amount is an amount effective for treating a lymphoproliferative disease in a subject in need thereof. In certain embodiments, the effective amount is an amount effective for preventing a lymphoproliferative disease in a subject in need thereof. In certain embodiments, the effective amount is an amount effective for treating a cancer (e.g., a lymphoid malignancy, a non-lymphoid malignancy, cancer associated with human immunodeficiency virus) in a subject in need thereof. In certain embodiments, the effective amount is an amount effective for preventing a cancer (e.g., a lymphoid malignancy, a non-lymphoid malignancy, cancer associated with human immunodeficiency virus) in a subject in need thereof. In certain embodiments, the effective amount is an amount effective for treating chronic active EBV infection (CAEBV) in a subject in need thereof. In certain embodiments, the effective amount is an amount effective for preventing chronic active EBV infection (CAEBV) in a subject in need thereof. In certain embodiments, the effective amount is an amount effective for treating multiple sclerosis (MS) in a subject in need thereof. In certain embodiments, the effective amount is an amount effective for preventing multiple sclerosis (MS) in a subject in need thereof. In certain embodiments, the effective amount is an amount effective for reducing the risk of developing a disease (e.g., a disease associated with Epstein-Barr virus (EBV), Kaposi's sarcoma-associated herpesvirus (KSHV)) (e.g., a proliferative disease (e.g., a lymphoproliferative disease, cancer (e.g., a lymphoid malignancy, a non-lymphoid malignancy, cancer associated with human immunodeficiency virus)), chronic active EBV infection (CAEBV), multiple sclerosis (MS)) in a subject in need thereof. In certain embodiments, the effective amount is an amount effective for inhibiting cell proliferation or promoting apoptosis in a subject in need thereof. In certain embodiments, the effective amount is an amount effective for inducing the lytic phase of a virus (e.g., Epstein-Barr virus (EBV), Kaposi's sarcoma-associated herpesvirus (KSHV)) in a subject in need thereof. In certain embodiments, the effective amount is an amount effective for reactivating a virus (e.g., Epstein-Barr virus (EBV), Kaposi's sarcoma-associated herpesvirus (KSHV)) in a subject in need thereof.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present in vivo.

Pharmaceutical compositions described herein can be prepared by any method known in the art of pharmaceutics. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents or fillers, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents or fillers include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, starches (such as dry starch, cornstarch), sugars (such as powdered sugar), calcium trisulfate, carboxymethylcellulose calcium, dextrate, dextrin, dextrose, fructose, lactitol, lactose, magnesium carbonate, magnesium, maltitol, maltodextrin, maltose, sucrose, glucose, mannitol, silicic acid, xylitol, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary disintegrating agents or disintegrants include agar, algin, alginic acid, sodium alginate, silicates, sodium carbonate, calcium carbonate, carboxymethylcellulose, cellulose, clay, colloidal silicon dioxide, croscarmellose sodium, crospovidone, rubber, magnesium silicate, methylcellulose, potassium krillin, hydroxypropylcellulose (e.g., low substituted Hydroxypropylcellulose), crosslinked polyvinylpyrrolidone, hydroxypropylcellulose, and starch (e.g., sodium glycolate starch, potato or tapioca starch).

Exemplary binding agents include starch (e.g., glycolate starch, cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include agar, ethyl oleate, ethyl laurate, glycerin, blyceryl palmitostearate, magnesium oxide, magnesium stearate, mannitol, poloxamer, glycol, sodium stearyl, sorbitol, zinc stearate, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

In some embodiments, injectable preparations of the compositions disclosed herein are in the form of a ready-to-use (“RTU”) preparation that can be directly administered to a subject. In some embodiments, the RTU preparation is a suspension. In some embodiments, the RTU preparation is a solution. In some embodiments, the RTU preparation is an emulsion. In some embodiments, injectable preparations of the compositions disclosed herein are in the form of a solid that is reconstituted prior to administration. In some embodiments, the solid is a lyophilized solid. In some embodiments, injectable preparations of the compositions disclosed herein are in the form of a liquid or suspension that is diluted prior to administration.

In some embodiments, the pharmaceutical compositions disclosed herein comprise a bulking agent. Bulking agents can be used, e.g., to improve the appearance of a solid composition, to provide visible “bulk” to demonstrate product quality or to facilitate preparation, e.g., of a solid composition prepared for reconstitution prior to administration. Bulking agents can be used for low dose (high potency) drugs that do not have the necessary bulk to support their own structure or provide a visible composition in a unit dosage form. Bulking agents are used in lyophilized formulations. Bulking agents provide a desirable structure for a lyophilized cake comprising pores that provide the means for vapor to escape from the product during lyophilization cycles, and facilitate dissolution on reconstitution. In some embodiments, the bulking agent is mannitol, lactose, sucrose, dextran, trehalose, povidone, dextran, glycine, isoleucine, methionine, or a cyclodextrin (e.g., (2-hydroxypropyl)-β-cyclodextrin).

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 ug and 1 ug, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, in inhibiting cell proliferation or promoting apoptosis in a subject in need thereof, in inducing the lytic phase of a virus (e.g., Epstein-Barr virus (EBV), Kaposi's sarcoma-associated herpesvirus (KSHV)) in a subject in need thereof, and/or in reactivating a virus (e.g., Epstein-Barr virus (EBV), Kaposi's sarcoma-associated herpesvirus (KSHV)) in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNA, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., a disease associated with Epstein-Barr virus (EBV), Kaposi's sarcoma-associated herpesvirus (KSHV)) (e.g., a proliferative disease (e.g., a lymphoproliferative disease, cancer (e.g., a lymphoid malignancy, a non-lymphoid malignancy, cancer associated with human immunodeficiency virus)), chronic active EBV infection (CAEBV), multiple sclerosis (MS)). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., a disease associated with Epstein-Barr virus (EBV), Kaposi's sarcoma-associated herpesvirus (KSHV)) (e.g., a proliferative disease (e.g., a lymphoproliferative disease, cancer (e.g., a lymphoid malignancy, a non-lymphoid malignancy, cancer associated with human immunodeficiency virus)), chronic active EBV infection (CAEBV), multiple sclerosis (MS)) in a subject in need thereof. In certain embodiments, the kits are useful for preventing a disease (e.g., a disease associated with Epstein-Barr virus (EBV), Kaposi's sarcoma-associated herpesvirus (KSHV)) (e.g., a proliferative disease (e.g., a lymphoproliferative disease, cancer (e.g., a lymphoid malignancy, a non-lymphoid malignancy, cancer associated with human immunodeficiency virus)), chronic active EBV infection (CAEBV), multiple sclerosis (MS)) in a subject in need thereof. In certain embodiments, the kits are useful for reducing the risk of developing a disease (e.g., a disease associated with Epstein-Barr virus (EBV), Kaposi's sarcoma-associated herpesvirus (KSHV)) (e.g., a proliferative disease (e.g., a lymphoproliferative disease, cancer (e.g., a lymphoid malignancy, a non-lymphoid malignancy, cancer associated with human immunodeficiency virus)), chronic active EBV infection (CAEBV), multiple sclerosis (MS)) in a subject in need thereof. In certain embodiments, the kits are useful for inhibiting cell proliferation or promoting apoptosis in a subject in need thereof. In certain embodiments, the kits are useful for inducing the lytic phase of a virus (e.g., Epstein-Barr virus (EBV), Kaposi's sarcoma-associated herpesvirus (KSHV)) in a subject in need thereof. In certain embodiments, the kits are useful for reactivating a virus (e.g., Epstein-Barr virus (EBV), Kaposi's sarcoma-associated herpesvirus (KSHV)) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., a disease associated with Epstein-Barr virus (EBV), Kaposi's sarcoma-associated herpesvirus (KSHV)) (e.g., a proliferative disease (e.g., a lymphoproliferative disease, cancer (e.g., a lymphoid malignancy, a non-lymphoid malignancy, cancer associated with human immunodeficiency virus)), chronic active EBV infection (CAEBV), multiple sclerosis (MS)) in a subject in need thereof. In certain embodiments, the kits and instructions provide for preventing a disease (e.g., a disease associated with Epstein-Barr virus (EBV), Kaposi's sarcoma-associated herpesvirus (KSHV)) (e.g., a proliferative disease (e.g., a lymphoproliferative disease, cancer (e.g., a lymphoid malignancy, a non-lymphoid malignancy, cancer associated with human immunodeficiency virus)), chronic active EBV infection (CAEBV), multiple sclerosis (MS)) in a subject in need thereof. In certain embodiments, the kits and instructions provide for reducing the risk of developing a disease (e.g., a disease associated with Epstein-Barr virus (EBV), Kaposi's sarcoma-associated herpesvirus (KSHV)) (e.g., a proliferative disease (e.g., a lymphoproliferative disease, cancer (e.g., a lymphoid malignancy, a non-lymphoid malignancy, cancer associated with human immunodeficiency virus)), chronic active EBV infection (CAEBV), multiple sclerosis (MS)) in a subject in need thereof. In certain embodiments, the kits and instructions provide for inhibiting cell proliferation or promoting apoptosis in a subject in need thereof. In certain embodiments, the kits and instructions provide for inducing the lytic phase of a virus (e.g., Epstein-Barr virus (EBV), Kaposi's sarcoma-associated herpesvirus (KSHV)) in a subject in need thereof. In certain embodiments, the kits and instructions provide for reactivating a virus (e.g., Epstein-Barr virus (EBV), Kaposi's sarcoma-associated herpesvirus (KSHV)) in a subject in need thereof. A kit described herein may include one or more additional pharmaceutical agents described herein as a separate composition.

In another aspect, the present disclosure provides a kit comprising:

a therapeutically effective amount of a dual HDAC3 and HDAC8 targeting agent, or a pharmaceutically acceptable salt thereof; and

instructions for its use.

In another aspect, the present disclosure provides a kit comprising:

a pharmaceutical composition comprising:
a therapeutically effective amount of a dual HDAC3 and HDAC8 targeting agent, or a pharmaceutically acceptable salt thereof; and
a therapeutically effective amount of an antiviral agent, or a pharmaceutically acceptable salt thereof; and

instructions for its use.

In another aspect, the present disclosure provides a kit comprising:

a pharmaceutical composition comprising:
a therapeutically effective amount of a dual HDAC3 and HDAC8 targeting agent, or a pharmaceutically acceptable salt thereof; and
a therapeutically effective amount of an agent capable of sensitizing or potentiating induction of the lytic phase of the virus, or a pharmaceutically acceptable salt thereof; and instructions for its use.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: YX968 Reactivates EBV Lytic Cycle

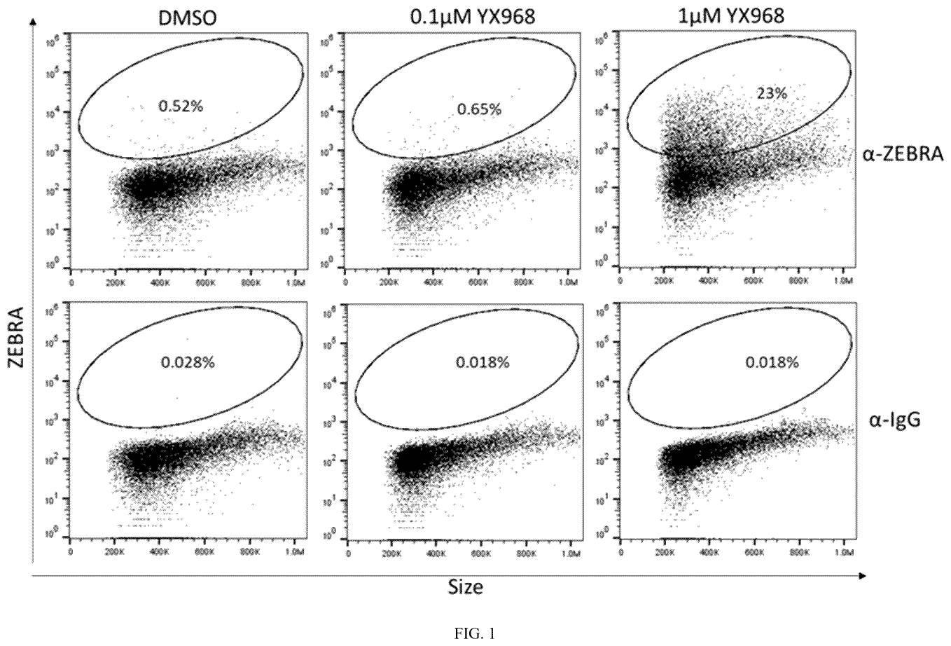

EBV-positive HH514-16 Burkitt lymphoma cell line was exposed to varying concentrations of YX968 or DMSO (0.1%) for 24 hours. Cells were harvested, fixed and permeabilized, stained with a monoclonal antibody to the viral latent-to-lytic switch protein ZEBRA (Bam Z EBV Replication Activator), and examined by flow cytometry. ZEBRA is an early lytic protein of EBV, encoded by BZLF1, that drives the lytic cascade. Cells were stained in parallel with an isotype-matched control antibody for placement of gates. The results show that treatment of cells with YX968 resulted in increased levels of ZEBRA, indictive of EBV switching to lytic phase (FIG. 1).

### Example 2: YX968 Induces a Late Lytic Gene of EBV

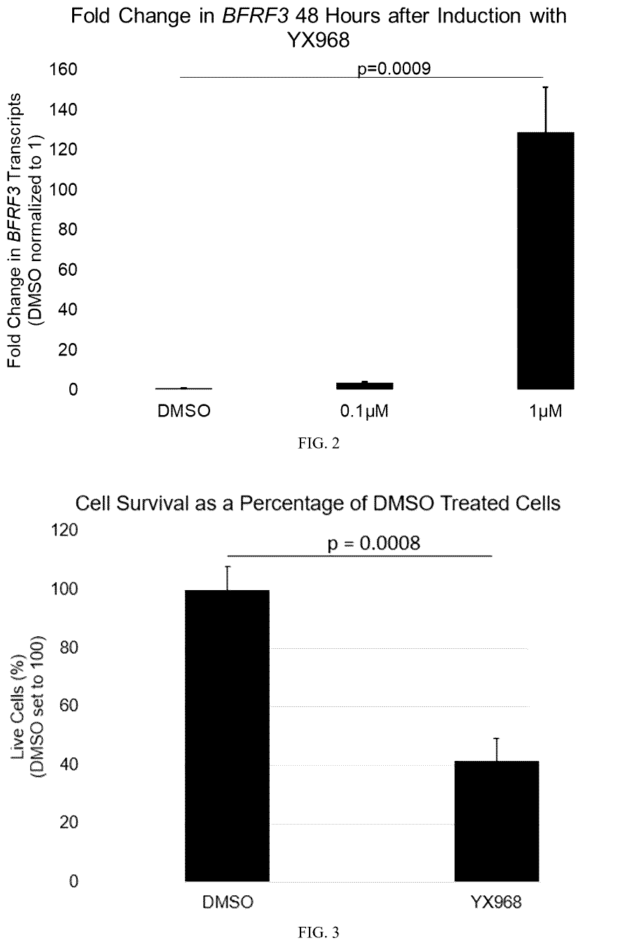

EBV-positive HH514-16 Burkitt lymphoma cell line was exposed to varying concentrations of YX968 or DMSO (0.1%) for 48 hours. Cells were harvested, RNA was extracted, RNA was converted to cDNA, and qPCR was performed. Expression of BFRF3, a late lytic gene, is increased (FIG. 2). The increase in BFRF3 indicates that there is no abortive lytic cycle, but rather that the lytic cycle is proceeding to completion. Progression of the lytic cycle to late lytic gene expression indicates availability of the early gene product needed to activate ganciclovir. N=3 biological replicates.

### Example 3: Burkitt Lymphoma Cells Die After Exposure to YX968

Activation of the EBV lytic phase is known to cause cell death. HH514-16 cells were treated for 72 hours with 1 μM YX968 or DMSO (0.1%) as a negative control. There were significantly fewer cells left alive in the YX968 treated condition versus the DMSO treated condition after the incubation period (FIG. 3). This indicates that YX968 can be used to kill cancer cells. N=2 biological replicates.

### Example 4: Ganciclovir Inhibits the Replication of the EBV Genome After the Lytic Cycle has Been Started by YX968

EBV-positive HH514-16 Burkitt lymphoma cell line was exposed to DMSO (0.1%) or 1 μM YX968 for 18 hours. Ganciclovir was then added at a concentration of 40 μM. Cells were then centrifuged, DNA was extracted, and qPCR for BALF5—a single copy EBV gene—was then used to determine the viral load of EBV in the cells. The ribosomal gene 18S was used as the control to normalize for the number of cells collected. The viral load of EBV was significantly lower when ganciclovir was added (FIG. 4). This indicates that ganciclovir can be used as an anti-viral agent in conjunction with YX968 to reduce the number of infectious virions released post-lytic induction of EBV-positive cancer cells. N=2 biological replicates.

### Example 5: KSHV RTA Transcripts Increase After Treatment with YX968

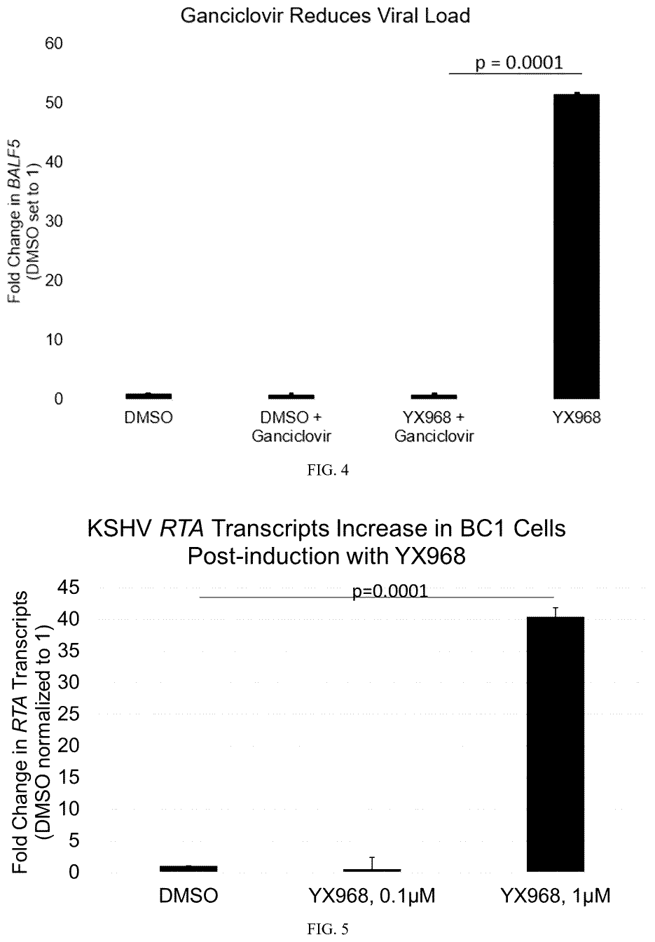

The lymphoma cell line BC1 is latently infected with KSHV. BC1 cells were treated with varying concentrations of YX968 or DMSO as a negative control. RNA was collected from cells, converted to cDNA, and assayed by qPCR. The immediate early gene RTA increases in expression 24 hours post-treatment with 1 μM YX968 (FIG. 5). This indicates the initialization of the lytic phase of KSHV. YX968 may therefore also be used for oncolytic therapy of KSHV-infected cancers. N=3 biological replicates.

### Example 6: Overexpression of HDAC2 Causes a Decrease in the Percentage of Lytic Cells Induced by YX968, Whereas Overexpression of HDAC1 Does Not

EBV-positive HH514-16 Burkitt lymphoma cell line was electroporated to introduce plasmids expressing either HDAC1-FLAG or FLAG-HDAC2. Twenty-four hours later, the cells were treated with 1 μM YX968 or 3 mM NaB (sodium butyrate). Twenty-four hours after treatment, cells were harvested and fixed. Cells were immunostained with antibodies against ZEBRA and FLAG. As measured by flow cytometry with staining using a ZBERA antibody, the cell population overexpressing HDAC1 (FLAG positive) had no significant change in the number of cells that expressed ZEBRA versus the cell population not overexpressing HDAC1 (FLAG negative). This is in contrast to cells overexpressing HDAC2 (FLAG positive). Cells overexpressing HDAC2 are less likely to express ZEBRA post-induction with either YX968 or NaB than cells which do not overexpress HDAC2. This indicates that HDAC2 may be inhibited by YX968, and this may be part of the molecular mechanism by which YX968 causes lytic reactivation of EBV. N=3 biological replicates.

### Example 7: Acetylation of H3K27 Increases at the BZLF1 Promoter Post-Addition of YX968

EBV-positive HH514-16 Burkitt lymphoma cell line was exposed to DMSO (0.1%) or 1 μM YX968 or 3 mM NaB for 12 hours. Cells were then crosslinked with formaldehyde. The fixed cells were then subjected to a chromatin immunoprecipitation assay using the SimpleChIP® Enzymatic Chromatin IP Kit with Magnetic Beads (Cell Signaling Technologies). Immunoprecipitation was achieved with the H3K27Ac antibody (D5E4; Cell Signaling Technologies). After digestion of proteins and reversal of crosslinks, DNA was isolated and amplified with primers targeting the BZLF1 promoter. Ct values were derived from qPCR. Fold enrichment was calculated with respect to control IgG. There was a statistically significant increase in the amount of acetylated H3K27 at the BZLF1 promoter. This indicates that one part of the mechanism by which YX968 increases the expression of BZLF1 could be through the increase in activating histone marks such as H3K27Ac at the BZLF1 promoter. N=3 technical replicates, representative of 3 biological replicates.

### Example 8: Combining YX968 and Tazemetostat Leads to a Greater Increase in Lytic Cells than Treatment with YX968 Alone

EBV-positive HH514-16 Burkitt lymphoma cell line was exposed to the EZH2 inhibitor tazemetostat or DMSO (0.1%) for 4 hours. Cells were then divided into two batches wherein one was subjected to additional DMSO (0.1%) and the other was subjected to YX968 for 24 hours. Cells were then harvested, fixed, immunostained with an antibody against ZEBRA, and subjected to flow cytometry analysis. Cells treated with DMSO twice or tazemetostat and DMSO did not induce beyond the background level of cells that spontaneously express ZEBRA. A significantly greater percentage of cells treated with YX968 and tazemetostat expressed ZEBRA than those treated with YX968 alone. N=3 biological replicates.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

1. A method of treating or preventing a disease associated with a virus in a subject in need thereof, the method comprising administering to the subject in need thereof a therapeutically effective amount of a dual HDAC3 and HDAC8 targeting agent, or a pharmaceutically acceptable salt thereof.

2. The method of claim 1, further comprising administering to the subject in need thereof a therapeutically effective amount of an antiviral agent, or a pharmaceutically acceptable salt thereof and/or a therapeutically effective amount of an agent capable of sensitizing or potentiating induction of the lytic phase of the virus, or a pharmaceutically acceptable salt thereof.

3-18. (canceled)

19. The method of claim 1, wherein the dual HDAC3 and HDAC8 targeting agent is YX968, or a pharmaceutically acceptable salt thereof.

20. The method of claim 2, wherein the antiviral agent is ganciclovir, cidofovir, acyclovir, or foscarnet, or a pharmaceutically acceptable salt thereof.

21. (canceled)

22. The method of claim 2, wherein the additional agent capable of sensitizing or potentiating induction of the lytic phase of the virus is an EZH2 inhibitor.

23. The method of claim 22, wherein the additional agent capable of sensitizing or potentiating induction of the lytic phase of the virus is tazemetostat.

24. The method of claim 1, further comprising administering to the subject in need thereof a therapeutically effective amount of an additional agent capable of inducing the lytic phase of the virus, or a pharmaceutically acceptable salt thereof, wherein the additional agent capable of inducing the lytic phase of the virus is an HDAC inhibitor, a DNA methyltransferase inhibitor, a phorbol ester, a hypoxia-mimicking agent, or an immunoglobulin.

25-26. (canceled)

27. The method of claim 24, wherein the additional agent capable of inducing the lytic phase of the virus is sodium butyrate, valproic acid, romidepsin, MS-275, apicidin, oxamflatin, Scriptaid, panobinostat, or nanatinostat.

28. The method of claim 1, wherein the virus is a herpesvirus.

29. (canceled)

30. The method of claim 1, wherein the virus is Epstein-Barr virus (EBV) or Kaposi's sarcoma-associated herpesvirus (KSHV).

31-32. (canceled)

33. The method of claim 1, wherein the disease is a proliferative disease selected from a lymphoproliferative disease, a cancer, chronic active EBV infection (CAEBV), and multiple sclerosis (MS).

34. (canceled)

35. The method of claim 33, wherein the lymphoproliferative disease is iatrogenic lymphoproliferative disorder, iatrogenic lymphoma, post-transplant lymphoproliferative disorder (PTLD), post-transplant lymphoma, Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cell carcinoma, AIDS-related lymphoma, chronic active EBV infection (CAEBV), hemophagocytic lymphohistiocytosis, EBV-positive gastric carcinoma, or multicentric Castleman's disease.

36. (canceled)

37. The method of claim 33, wherein the cancer is:
a lymphoid malignancy selected from iatrogenic lymphoma, post-transplant lymphoma, Burkitt lymphoma, B-cell non-Hodgkin lymphoma, diffuse large B-cell lymphoma (DLBCL), central nervous system (CNS) lymphoma, NK/T-cell lymphoma, T cell non-Hodgkin lymphoma, Hodgkin's lymphoma, and a primary effusion lymphoma (PEL);
a non-lymphoid malignancy selected from EBV-positive gastric carcinoma and nasopharyngeal cell carcinoma;
a cancer associated with human immunodeficiency virus selected from oral hairy leukoplakia, central nervous system (CNS) lymphoma, or AIDS-related lymphoma; or
Kaposi's sarcoma.

38-45. (canceled)

46. A method of inhibiting cell proliferation or promoting apoptosis in a subject in need thereof, the method comprising administering to the subject in need thereof an effective amount of a dual HDAC3 and HDAC8 targeting agent, or a pharmaceutically acceptable salt thereof.

47. A method of inducing a lytic phase of a virus in a subject in need thereof, the method comprising administering to the subject in need thereof an effective amount of a dual HDAC3 and HDAC8 targeting agent, or a pharmaceutically acceptable salt thereof.

48-73. (canceled)

74. The method of claim 1, further comprising administering to the subject in need thereof one or more additional pharmaceutical agents and/or radiation.

75. The method of claim 74, wherein the one or more additional pharmaceutical agents are chemotherapeutic agents and/or PARP inhibitors.

76-77. (canceled)

78. The method of claim 1, wherein:
the subject in need thereof has been administered an immunosuppressive or immunomodulatory agent selected from corticosteroids (e.g., prednisone (Deltasone, Orasone), budesonide (Entocort EC), prednisolone (Millipred)), Janus kinase inhibitors (e.g., tofacitinib (Xeljanz)), calcineurin inhibitors (e.g., cyclosporine (Neoral, Sandimmune, SangCya), tacrolimus (Astagraf XL, Envarsus XR, Prograf)), mTOR inhibitors (e.g., sirolimus (Rapamune), everolimus (Afinitor, Zortress), inosine monophosphate dehydrogenase (IMDH) inhibitors (e.g., azathioprine (Azasan, Imuran), leflunomide (Arava), mycophenolate (CellCept, Myfortic), biologics (e.g., antibody, monoclonal antibody) (e.g., abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta)), and antimetabolites (e.g., methotrexate); and/or
the subject in need thereof has been administered one or more of T cell suppressive therapy and anti-TNF alpha therapy, wherein the T cell suppressive therapy is antithymocyte globulin or recombinant antithymocyte globulin therapy.

79-89. (canceled)

90. The method of claim 1, wherein the subject in need thereof comprises one or more of EBV-positive cancer cells, EBV-positive pre-cancerous cells, KSHV-positive cancer cells, and KSHV-positive pre-cancerous cells.

91. The method of claim 90, wherein the EBV or KSHV is in a latent lytic state or abortive lytic state.

92-112. (canceled)
